(19) United States
(12) Patent Application Publication
(10) Pub. No.: US 2019/0311789 A1
(43) Pub. Date: Oct. 10, 2019

(54) COMPUTER IMPLEMENTED DISCOVERY OF BIOMARKERS FOR BLOOD BRAIN BARRIER DISRUPTION

(71) Applicant: WEST VIRGINIA UNIVERSITY, Morgantown, WV (US)

(72) Inventors: Taura L. BARR, Waynesburg, PA (US); Grant O'CONNELL, Morgantown, WV (US)

(21) Appl. No.: 16/339,173

(22) PCT Filed: Oct. 3, 2017
(86) PCT No.: PCT/US17/54946

§ 371 Date: Apr. 3, 2019

Related U.S. Application Data
(60) Provisional application No. 62/403,366, filed on Oct. 3, 2016.

(51) Int. Cl. G16H 10/40; G16B 20/00; G16H 50/30

(52) U.S. Cl. CPC G16H 10/40; A61B 34/10; G16H 50/30; G16B 20/00

(57) ABSTRACT

Provided herein are computer implemented methods of evaluating, detecting, and identifying biomarkers of blood brain barrier disruption. Also provided herein are kits and methods for detecting blood brain barrier disruption in a subject.

### CROSS-REFERENCE

This application claims the benefit of U.S. Provisional Patent Application No. 62/403,366, filed on Oct. 3, 2016, which is herein incorporated by reference in its entirety.

### GOVERNMENT SUPPORT

This invention was made with government support under Project No. 5P20GM109098, Sub-project ID 5375, awarded by the National Institutes of Health. The government has certain rights in the invention.

### BACKGROUND

Stroke can be defined as the interruption of blood flow to brain tissue. Specifically, strokes can occur when there is an interruption in blood flow by the blockage or rupture of a blood vessel that serves the brain. The administration of thrombolytic agents can be an effective treatment for strokes, however, thrombolytic agents such as tissue plasminogen activator (tPA) must be administered within a finite period. Thus, early and rapid diagnosis of stroke can be critical for treatment. In many cases, expert neurological assessment is often needed for accurate diagnosis of ischemic stroke. In institutions where advanced neuroimaging is available, CT or MRI can be often used as a diagnostic and/or confirmatory tool. However, most health care institutions do not have access to advanced imaging technologies or the expertise required to make a confirmatory diagnosis of strokes. Ideally, it would be desirable to provide additional tools to diagnose strokes in a time sensitive manner. Evaluating the expression patterns of biomarkers in peripheral blood can allow for the diagnosis of stroke in a time-sensitive and bedside manner.

The post-acute inflammatory milieu which develops following ischemic stroke can promote delayed disruption of the blood brain barrier (BBB) within the lesion and in surrounding tissue. Elevated activities of matrix metalloproteinases and trafficking of peripheral immune cells into the brain parenchyma increases the likelihood of hemorrhagic transformation and vasogenic edema, complications associated with poor post-injury outcome. Thrombolytic intervention via rTPA can increase the risk of such adverse events and worsen outcome in the subset patients who experience complications. Early identification of patients at risk for post-stroke BBB disruption could allow for clinicians to make more informed decisions regarding the administration of thrombolytic therapies, and ultimately improve clinical managements. Unfortunately, the tools available to clinicians to identify such patients in the acute phase of care can be limited.

Currently, one of the most sensitive methods to clinically detect early changes in BBB permeability is contrast MRI using a gadolinium based contrast agent such as gadolinium-diethylene triamine penta-acetic acid (Gd-DTPA). Because the intact BBB can be largely impermeable to Gd-DTPA, hyperintense post-contrast enhancement of the CSF space on fluid-attenuated inversion recovery (FLAIR) can be indicative of BBB disruption, and is known as hyperintense acute reperfusion injury marker (HARM). Ischemic stroke patients who exhibit HARM in the acute phase of care can be more likely to later develop edema or undergo hemorrhagic transformation. While such imaging techniques can provide valuable information which can be used to guide clinical care decisions, most healthcare facilities lack dedicated MRI facilities to perform acute triage. Because of this, the identification of rapidly measurable peripheral blood biomarkers which can provide similar diagnostic information could prove invaluable in the acute phase of care.

### SUMMARY

Disclosed herein are methods that can comprise performing, using a computer processor, an algorithm on a biological sample from a subject to generate a fitness score for a chromosome of data. The subject may be previously diagnosed with a blood-brain barrier disruption as determined by contrast MRI. In some cases, a computer processor can execute instructions to perform a functional classification enrichment analysis. Also disclosed herein are methods that can comprise performing multiple iterations of an algorithm until a fitness score exceeds a termination cutoff. Also disclosed herein are methods that can comprise compiling a profile. A profile can comprise at least one biomarker that can be involved in chemotaxis as determined by functional classification enrichment analysis. In some embodiments, an algorithm can comprise a machine learning algorithm. In some embodiments, a machine learning can comprise a deep learning algorithm. In some embodiments, an algorithm can comprise analyzing an initial panel of at least about 10,000 genes. In some embodiments, a machine learning algorithm can comprise genetic algorithm k-neared neighbors. In some embodiments, a termination cutoff can be about 0.85. In some embodiments, a chromosome of data has a chromosome length of at least about 10.

Also disclosed herein are systems for detecting a blood-brain barrier disruption in a subject that can comprise a memory that stores executable instructions. Also disclosed herein are systems for detecting a blood-brain barrier disruption in a subject that can comprise a computer processor that can executes instruction to perform a method described herein. In some embodiments, a system can further comprise an integrated storage device. In some embodiments, a system can be configured to communicate with a database for performing functional classification enrichment analysis.

Also disclosed herein are kits for assessing blood-brain barrier disruption in a subject that can comprise a probe for measuring a presence of a panel of biomarkers in a biological sample obtained from a subject. A panel of biomarkers can comprise a nucleic acid. A probe can hybridize to a nucleic acid in a biological sample. Also disclosed herein are kits for assessing blood-brain barrier disruption in a subject that can comprise a detecting reagent to examine hybridization of a probe to a nucleic acid. A panel of biomarkers can comprise one or more biomarkers selected from the group consisting of: RBP7, CCDC149, DDIT4, E2F3, and ADAM15. In some embodiments, a kit can further comprise instructions for use. In some embodiments, a panel of biomarkers can comprise at least two biomarkers. In some embodiments, a panel of biomarkers can comprise RBP7, CCDC149, DDIT4, E2F3, and ADAM15. In some embodiments, a panel of biomarkers can further comprise LAIR2, IL-8, CXCL5, LY96, HPSE, ACN9, TMEM176B, BIRC2, EMR2, DUSP1, HSPA1B, RNASE2, IDI1, SCOC, FAM65A, CD14, F2RL1, PCMTD1, SMEK2, or SDPR. In some embodiments, a panel of biomarkers can comprise LAIR2, IL-8, CXCL5, LY96, and HPSE. In some embodiments, a panel of biomarkers can comprise LAIR2, RBP7, CCDC149, DDIT4, E2F3, ADAM15, LAIR2, IL-8, CXCL5, LY96, and HPSE. In some embodiments, a kit can further comprise a communication medium that can be configured to communicate hybridization of a probe to a nucleic acid. In some embodiments, a communication medium can be an electronic medium.

Also disclosed herein are methods that can comprise determining a presence of a panel of biomarkers in a biological sample obtained from a subject using an assay. A subject can be a subject having blood brain barrier disruption. A subject can be a subject suspected of having blood brain barrier disruption. A panel of biomarkers can comprise one or more biomarkers selected from the group consisting of: RBP7, CCDC149, DDIT4, E2F3, and ADAM15. Also disclosed herein are methods that can comprise comparing a presence of a panel of biomarkers in a biological sample obtained from a subject to a reference derived from one or more control samples. In some embodiments, a panel of biomarkers can comprise at least two biomarkers. In some embodiments, a panel of biomarkers can comprise RBP7, CCDC149, DDIT4, E2F3, and ADAM15. In some embodiments, a panel of biomarkers can further comprise LAIR2, IL-8, CXCL5, LY96, HPSE, ACN9, TMEM176B, BIRC2, EMR2, DUSP1, HSPA1B, RNASE2, IDI1, SCOC, FAM65A, CD14, F2RL1, PCMTD1, SMEK2, or SDPR. In some embodiments, a panel of biomarkers can comprise LAIR2, RBP7, CCDC149, DDIT4, E2F3, ADAM15, LAIR2, IL-8, CXCL5, LY96, and HPSE. In some embodiments, one or more biomarkers can comprise ribonucleic acid. In some embodiments, one or more biomarkers can comprise a gene that can be involved in chemotaxis. In some embodiments, a subject can be suspected of having a stroke. In some embodiments, one or more control samples can be from one or more control subjects. In some embodiments, one or more control subjects can be stroke subjects. In some embodiments, stroke subjects can be ischemic stroke subjects. In some embodiments, one or more control subjects can be nonstroke subjects. In some embodiments, a reference was determined after one or more control subjects were administered a contrast agent. In some embodiments, a contrast agent can comprise a gadolinium-based contrast agent. In some embodiments, a gadolinium-based contrast agent can comprise gadolinium-diethylene triamine penta-acetic acid (Gd-DTPA). In some embodiments, one or more control subjects were diagnosed with a blood brain barrier disruption or a risk of a blood-brain barrier disruption. In some embodiments, a presence can comprise a level of a panel of biomarkers. In some embodiments, a method can further comprise assessing a blood brain barrier disruption in a subject. In some embodiments, an assessing can comprise determining a presence of a blood brain barrier disruption. In some embodiments, an assessing can comprise determining a risk of a blood brain barrier disruption. In some embodiments, an assessing can comprise determining an absence of a blood brain barrier disruption. In some embodiments, a panel of biomarkers can be at least about 1.5 fold higher in a subject relative to a reference. In some embodiments, a panel of biomarkers can be at least about 1.5 fold lower in a subject relative to a reference. In some embodiments, an assessing can be performed with a sensitivity of at least about 90%. In some embodiments, an assessing can be performed with a specificity of at least about 96%. In some embodiments, an assay can comprise hybridizing a probe to a panel of biomarkers or a portion thereof. In some embodiments, a method can further comprise detecting a hybridizing. In some embodiments, a probe can be a fluorescent probe. In some embodiments, a method can further comprise communicating a result through a communication medium when a probe hybridizes with a panel of biomarkers or a portion thereof. In some embodiments, a communication medium can comprise an electronic medium. In some embodiments, a biological sample can comprise whole blood, peripheral blood, or cerebrospinal fluid. In some embodiments, a biological sample can comprise cell-free nucleic acids.

Also disclosed herein are methods that can comprise determining a presence of a panel of biomarkers in a biological sample obtained from a subject. A subject can be a subject having stroke. A subject can be a subject suspected of having stroke. A determining can comprise using an assay. In some cases, a presence of a panel biomarkers can be indicative of hyperintense acute reperfusion marker (HARM) on fluid-attenuated inversion recovery (FLAIR) MRI. In some cases, a contrast agent can be administered to a subject. In some cases, a subject can be a subject having stroke. In some cases, a subject can be a subject suspected of having stroke. A panel of biomarkers can comprise one or more biomarkers selected from the group consisting of: LAIR2, RBP7, CCDC149, DDIT4, E2F3, and ADAM15. In some embodiments, a panel of biomarkers can comprise at least two biomarkers. In some embodiments, a panel of biomarkers can comprise LAIR2, RBP7, CCDC149, DDIT4, E2F3, and ADAM15. In some embodiments, a panel of biomarkers can further comprise IL-8, CXCL5, LY96, HPSE, ACN9, TMEM176B, BIRC2, EMR2, DUSP1, HSPA1B, RNASE2, IDI1, SCOC, FAM65A, CD14, F2RL1, PCMTD1, SMEK2, or SDPR. In some embodiments, a panel of biomarkers can comprise IL-8, CXCL5, LY96, and HPSE. In some embodiments, a panel of biomarkers can comprise LAIR2, RBP7, CCDC149, DDIT4, E2F3, ADAM15, IL-8, CXCL5, LY96, and HPSE. In some embodiments, a stroke can be an ischemic stroke. In some embodiments, a contrast agent can comprise a gadolinium-based contrast agent. In some embodiments, a gadolinium-based contrast agent can comprise gadolinium-diethylene triamine penta-acetic acid (Gd-DTPA). In some embodiments, a HARM can be severe HARM. In some embodiments, severe HARM can be indicative of a blood-brain barrier disruption. In some embodiments, a presence can comprise a level of a panel of biomarkers. In some embodiments, a method can further comprise comparing a presence of a panel of biomarkers to a reference. In some embodiments, a reference can be derived from one or more control samples. In some embodiments, a panel of biomarkers can be at least about 1.5 fold higher in a subject relative to a reference. In some embodiments, a panel of biomarkers can be at least about 1.5 fold lower in a subject relative to a reference. In some embodiments, a method can further comprise administering a therapeutic to a subject. In some embodiments, an assay can comprise hybridizing a probe to a panel of biomarkers or portions thereof. In some embodiments, a method can further comprise detecting a hybridizing. In some embodiments, a probe can be a fluorescent probe. In some embodiments, a method can further comprise communicating a result through a communication medium when a probe hybridizes with a panel of biomarkers or a portion thereof. In some embodiments, a communication medium can comprise an electronic medium. In some embodiments, a biological sample can comprise whole blood, peripheral blood, or cerebrospinal fluid. In some embodiments, a biological sample can comprise cell-free nucleic acids.

Also disclosed herein are methods that can comprise determining a presence of a panel of biomarkers in a biological sample obtained from a subject using an assay. In some cases, a method can comprise determining a profile for a subject. Also disclosed herein are methods that can comprise assessing a blood brain barrier disruption in a subject. In some cases, an assessing can be performed with a sensitivity of at least about 90%. In some cases, an assessing can be performed with a specificity of at least about 96%. In some embodiments, a panel of biomarkers can comprise at least two biomarkers. In some embodiments, a panel of biomarkers can comprise one or more biomarkers selected from the group consisting of: LAIR2, IL-8, CXCL5, RBP7, CCDC149, LY96, DDIT4, HPSE, E2F3, ADAM15, ACN9, TMEM176B, BIRC2, EMR2, DUSP1, HSPA1B, RNASE2, IDI1, SCOC, FAM65A, CD14, F2RL1, PCMTD1, SMEK2, and SDPR. In some embodiments, a panel of biomarkers can comprise LAIR2, RBP7, CCDC149, DDIT4, E2F3, ADAM15, IL-8, CXCL5, LY96, and HPSE. In some embodiments, a panel of biomarkers can comprise ribonucleic acid. In some embodiments, biomarkers can comprise a gene that can be involved in chemotaxis. In some embodiments, a method can further comprise comparing a profile to a reference. In some embodiments, one or more control samples can be from one or more control subjects. In some embodiments, a reference was determined after one or more control subjects were administered a contrast agent. In some embodiments, a contrast agent can comprise a gadolinium-based contrast agent. In some embodiments, a gadolinium-based contrast agent can comprise gadolinium-diethylene triamine penta-acetic acid (Gd-DTPA). In some embodiments, an assessing can comprise determining a presence of a blood brain barrier disruption. In some embodiments, an assessing can comprise determining a risk of a blood brain barrier disruption. In some embodiments, an assessing can comprise determining an absence of a blood brain barrier disruption. In some embodiments, a biological sample can comprise whole blood, peripheral blood, or cerebrospinal fluid. In some embodiments, a biological sample can comprise cell-free nucleic acids.

### INCORPORATION BY REFERENCE

All publications, patents, and patent applications mentioned in this specification are herein incorporated by reference in their entireties to the same extent as if each individual publication, patent, or patent application was specifically and individually indicated to be incorporated by reference in their entireties.

### BRIEF DESCRIPTION OF THE DRAWINGS

The novel features described herein are set forth with particularity in the appended claims. A better understanding of the features and advantages of the features described herein will be obtained by reference to the following detailed description that sets forth illustrative examples, in which the principles of the features described herein are utilized, and the accompanying drawings of which:

FIG. 1A shows the top exemplary genes identified by GA/kNN for prediction of post stroke BBB disruption. The most predictive genes ranked by GA/kNN, ordered by the number of times each was selected as part of a near-optimal solution.

FIG. 1B shows the combined ability of the expression levels of the top ranked exemplary genes to discriminate between patients who developed post-stroke severe HARM and those who did not using kNN in leave one out cross validation.

FIG. 1C shows the peripheral blood differential expression of the top ranked exemplary transcripts with fold change reported relative to mild HARM.

FIG. 1D shows a coordinate pattern of expression of the top ten exemplary genes plotted for each subject across both experimental groups.

FIG. 2 shows functional annotation enrichment. Biological processes enriched among the top 25 exemplary genes identified by GA/kNN as being predicative of severe HARM.

FIG. 3A shows the use of GA/kNN for the identification of genes with strong predictive ability. Following expression profiling, a small combination of genes (referred to as a chromosome or a chromosome of data) can be generated by random selection from the total pool of gene expression data. The predictive ability of the chromosome can be quantified as a fitness score, or the proportion of samples which the chromosome can correctly predict. A termination cutoff (minimum proportion of correct predications) determines the level of fitness required to pass evaluation. A chromosome which passes kNN evaluation can be identified as a near optimal solution and can be recorded, while a chromosome which fails evaluation undergoes mutation and can be re-evaluated. This process of mutation and re-evaluation can be repeated until the fitness score of the chromosome exceeds the termination cutoff.

FIG. 3B shows the ability of this chromosome to predict sample class evaluated using kNN. In this kNN evaluation, each sample can be plotted as a vector in an nth dimensional space, with the coordinates of each vector being comprised of the expression levels of the genes of the chromosome. The class of each sample can be predicted based on the majority class of the other samples which lie closest in Euclidian distance, which can be referred to the nearest neighbors.

FIG. 3C shows this process can be repeated multiple times (typically thousands) to generate a pool of heterogeneous near-optimal solutions.

FIG. 3D shows the predicative ability of each gene in the total pool of gene expression can be ranked according to the number of times it was part of a near-optimal solution.

FIG. 3E shows the collective predictive ability of the top ranked exemplary genes can then be tested via kNN in a leave one out cross validation.

FIG. 4 shows the identification of HARM on post-contrast FLAIR. The left panel depicts a pre-contrast FLAIR image from a subject. The right panel depicts a post-contrast FLAIR image from the same subject representative of what was identified as positive for HARM. Areas of HARM are indicated by a box.

FIG. 5 shows an exemplary computer implement workflow. Biomarkers from a peripheral blood sample from a subject can be detected using an assay. With the aid of a computer processor, a panel can compiled and a result can be communicated to the subject and/or stored onto storage means.

### DETAILED DESCRIPTION

### Overview

Provided herein are computer implemented methods and systems for identifying biomarkers that can be implicated in disruption of a blood-brain barrier (BBB). In some cases, a method can comprise: performing, using a computer processor, functional classification enrichment analysis on a biological sample from a subject to generate a fitness score for a chromosome of data. A subject can, in some instances, be a subject that was previously diagnosed with a blood-brain barrier disruption as determined by a method known in the art (e.g. contrast MRI). A computer processor as disclosed herein can execute instructions to perform a functional classification enrichment analysis. In some cases, multiple iterations of the functional classification enrichment analysis can be performed until a fitness score exceeds a termination cutoff. This analysis can be employed to compile a profile that can be predictive of incidence of a BBB disruption.

In some cases, a system as described herein can include a memory that can store instructions to perform a method described herein. The memory can be operatively connected to a computer processor that can execute instructions to perform a method described herein. A system can be configured to interact with and/or access a database. For example, a system can access a structural and/or functional database in order to analyze a biomarker. Such analysis can include grouping a biomarker according to a recited function.

Also provided herein are methods for assessing BBB disruption in a subject. A method can include determining in an assay a presence of one or more biomarkers in a biological sample. The presence of the one or more biomarkers can be compared to a reference that can be obtained from one or more control samples. In some instances, a control sample can include a sample from a control subject known to have a disruption in a BBB, a sample from a subject known to not have a disruption in a BBB, a sample from a stroke subject, a sample from a non stroke subject or a combination thereof. An assay can include detecting a presence of a single biomarker, or can include detecting of a plurality of biomarkers. In some cases, a presence of a biomarker can include a level of a biomarker. A presence or a level of a biomarker can be indicative of a disruption of a BBB in a subject. In some instances, a presence or an absence of a BBB disruption in a subject can be indicated by a presence or level of a biomarker. In some cases, a risk or a BBB disruption can be indicated by a presence or level of a biomarker. A presence or a level of a biomarker can be predictive of a positive or severe hyperintense acute reperfusion marker (HARM) on fluid-attenuated inversion recovery (FLAIR) MRI test. A presence or a level of a biomarker can be predictive of a no HARM on fluid-attenuated inversion recovery (FLAIR) MRI test. A presence or level of a biomarker can, in some cases, be indicative of a risk of developing a stroke (e.g. ischemic stroke). A determination or an assessment regarding a presence, absence, or risk of a condition can be performed with a high sensitivity and/or selectivity by modulation of the number and identity of biomarkers used in the assay.

### Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” can mean within an acceptable error range for the particular value as determined by one of ordinary skill in the art, which will depend in part on how the value is measured or determined, e.g., the limitations of the measurement system. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, within 5-fold, or within 2-fold, of a value. Where particular values are described in the application and claims, unless otherwise stated the term “about” meaning within an acceptable error range for the particular value should be assumed. Also, where ranges and/or subranges of values are provided, the ranges and/or subranges can include the endpoints of the ranges and/or subranges.

The term “subject”, “patient” or “individual” as used herein can encompass a mammal or a non-mammal. A mammal can be any member of the Mammalian class, including but not limited to a human, a non-human primates such as a chimpanzee, an ape or other monkey species; a farm animal such as cattle, a horse, a sheep, a goat, a swine; a domestic animal such as a rabbit, a dog (or a canine), and a cat (or a feline); a laboratory animal including a rodent, such as a rat, a mouse and a guinea pig, and the like. A non-mammal can include a bird, a fish and the like. In some embodiments, a subject can be a mammal. In some embodiments, a subject can be a human. In some instances, a human can be an adult. In some instances, a human can be a child. In some instances, a human can be age 0-17 years old. In some instances, a human can be age 18-130 years old. In some instances, a subject can be a male. In some instances, a subject can be a female. In some instances, a subject can be diagnosed with, or can be suspected of having, a condition or disease. In some instances a disease or condition can be disruption of a BBB. A subject can be a patient. A subject can be an individual. In some instances, a subject, patient or individual can be used interchangeably.

The term “stroke” can refer to a condition of poor blood flow in a brain in a subject. In some cases, a stroke can result in cell death in a subject. In some cases, a stroke can be an ischemic stroke. An ischemic stroke can be a condition in which a decrease or loss of blood in an area of a brain that can result in tissue damage or destruction. In some cases, a stroke can be a hemorrhagic stroke. A hemorrhagic stroke can be a condition in which bleeding in a brain or an area around a brain can result in tissue damage or destruction. In some cases, a stroke can result in a reperfusion injury. A reperfusion injury can include inflammation, oxidative damage, hemorrhagic transformation, and the like. In some cases, a stroke can result in a disruption of a blood-brain barrier. In some cases, a stroke may not result in a disruption of a blood-brain barrier.

The terms “biomarker” and “biomarkers” can be used interchangeably to refer to one or more biomolecules. In some cases, a biomarker can be a biomolecule associated with a disease. When associated with a disease, a biomarker can have a profile different under the disease condition compared to a non-disease condition. Biomarkers can be any class of biomolecules, including polynucleotides, polypeptides, carbohydrates and lipids. In some cases, a biomarker can be a polynucleotide. In some cases, a biomarker can be a polypeptide. A polynucleotide can be any type of nucleic acid molecule, including DNA, RNA, a hybridization thereof, or any combination thereof. For example, a polynucleotide can be cDNA, genomic DNA, mRNA, tRNA, rRNA, or microRNA. In some cases, a polynucleotide can be a cell-free nucleic acid molecule. In other cases a polynucleotide can be a cell-free nucleic acid molecule circulating in blood or a cellular nucleic acid molecule in a cell circulating in blood. A polypeptide or protein can be contemplated to include any fragments thereof, in particular, immunologically detectable fragments. A biomarker can also include one or more fragments of the biomarker having sufficient sequence such that it still possesses the same or substantially the same function as the full-size biomarker. An active fragment of a biomarker retains 100% of the activity of the full-size biomarker, or at least about 99%, 95%, 90%, 85%, 80% 75%, 70%, 65%, 60%, 55%, or at least 50% of its activity. In certain cases, an active fragment of a biomarker can be detectable (e.g., a polypeptide detectable by an antibody, or a polynucleotide detectable by an oligonucleotide). A biomarker associated with a disruption of a BBB can be a biomolecule associated with a disruption of a BBB. In some cases, a biomarker of BBB disruption can be a biomolecule associated with BBB, but not associated with other conditions. In some cases, a biomarker of BBB disruption can be a biomolecule associated with disruption of a BBB and other diseases or conditions.

### Methods

Provided herein are methods of assessing blood brain barrier disruption in a subject (e.g., a subject suspected of having a blood brain barrier disruption).

The post-acute inflammatory milieu which develops following ischemic stroke of a patient can promote delayed disruption of the blood brain barrier (BBB) within the lesion and in surrounding tissue. Elevated activities of matrix metalloproteinases and trafficking of peripheral immune cells into the brain parenchyma increases the likelihood of hemorrhagic transformation and vasogenic edema, complications associated with poor post-injury outcome. Thrombolytic intervention via rTPA can increase the risk of such adverse events and worsen outcome in the subset patients who experience complications. Early identification of patients at risk for post-stroke BBB disruption could allow for clinicians to make more informed decisions regarding the administration of thrombolytic therapies, and ultimately improve clinical management. Unfortunately, the tools available to clinicians to identify such patients in the acute phase of care can be limited.

Currently, one of the most sensitive methods to clinically detect early changes in BBB permeability is contrast MRI. Contrast MRI can include administering to a subject a contrast agent prior to, during, or after MRI imaging. Examples of contrast agents can include gadolinium contrast agents such as gadoterate (Dotarem, Clariscan), gadodiamide (Omniscan), gadobenate (MultiHance), gadopentetate (Magnevist), gadoteridol (ProHance), gadoversetamide (OptiMARK), gadobutrol (Gadovist [EU]/Gadavist [US]), gadopentetic acid dimeglumine (Magnetol), gadofosveset (Ablavar, formerly Vasovist), gadocoletic acid, gadomelitol, or gadomer 17; an iron oxide contrast agent; an iron platinum particle; a manganese compound; a barium compound such as barium sulfate; perflubron; a protein; a salt of any of these; and combinations of any of these. In some embodiments, a contrast agent can be a gadolinium-based contrast agent such as gadolinium-diethylene triamine penta-acetic acid (Gd-DTPA). Because the intact BBB can be largely impermeable to Gd-DTPA, hyperintense post-contrast enhancement of the CSF space on fluid-attenuated inversion recovery (FLAIR) can be indicative of BBB disruption, and is known as hyperintense acute reperfusion injury marker (HARM). Ischemic stroke patients who exhibit HARM in the acute phase of care are more likely to later develop edema or undergo hemorrhagic transformation. While such imaging techniques can provide valuable information which can be used to guide clinical care decisions, most healthcare facilities lack dedicated MRI facilities to perform acute triage. Because of this, the identification of rapidly measurable peripheral blood biomarkers which can provide similar diagnostic information could prove invaluable in the acute phase of care.

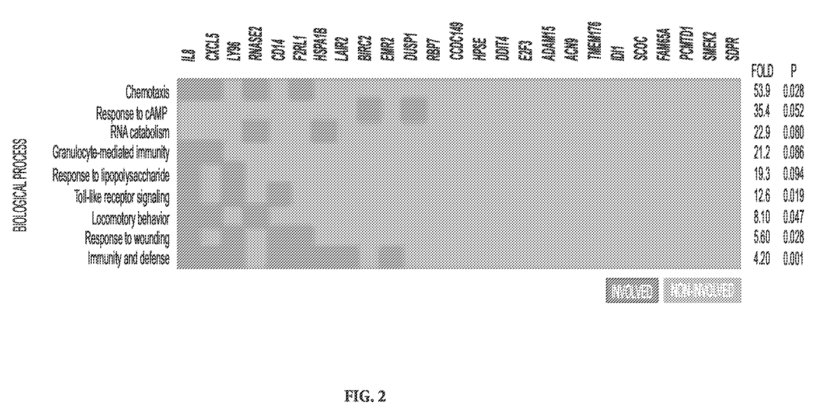

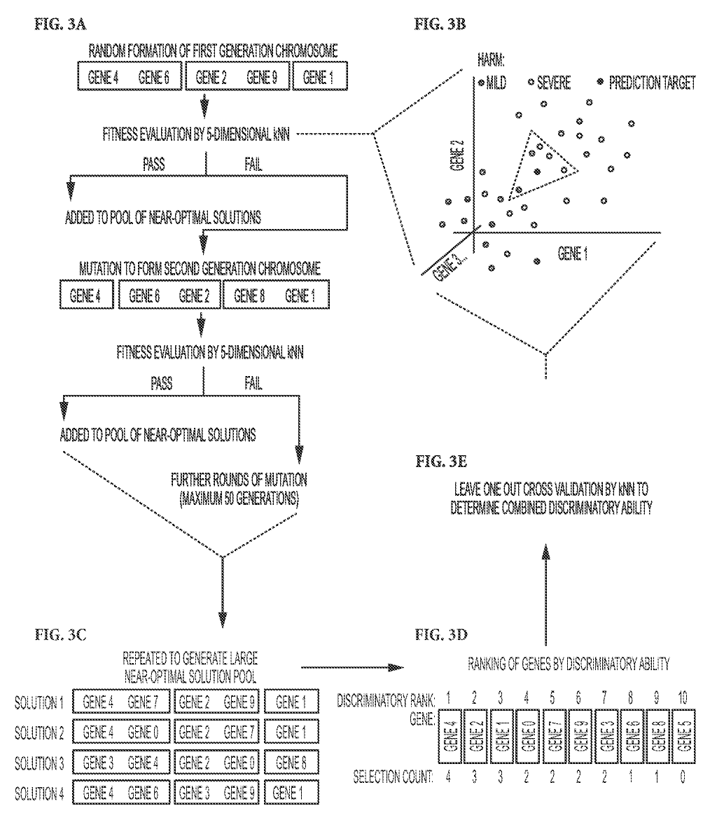

Because peripheral leukocyte populations play a major contributing role in the breakdown of the BBB, it may be possible that there can be early changes in the complexion peripheral immune system which predicate BBB disruption following ischemic stroke. It is well established the transcriptome of the peripheral immune system responds robustly and rapidly to ischemic injury, and it may be possible that the peripheral blood transcriptome may be a viable source of biomarkers which could be used to predict post-stroke BBB disruption. In some aspects, high throughput transcriptomics in tandem with a machine learning technique known as genetic algorithm k-neared neighbors (GA/kNN) can be used to identify a pattern of gene expression in peripheral blood which can be used to identify acute ischemic stroke with high levels of accuracy (REF). In this approach, gene expression data can be generated via microarray, and search heuristic known as genetic algorithm can be used to search for a combination of genes whose coordinate expression levels can optimally discriminate between experimental groups using a non-parametric classification method known as k-nearest neighbors (FIG. 3A).

The methods disclosed herein can be used to predict a disruption of a BBB. In some cases, a biomarker for a disruption of a BBB can be used to distinguish a subject displaying HARM from a subject not displaying HARM. In some cases, a biomarker for a disruption of a BBB can be used to distinguish a subject displaying mild HARM from a subject not displaying HARM. In some cases, a biomarker for a disruption of a BBB can be used to distinguish a subject displaying intermediate HARM from a subject not displaying HARM. In some cases, a biomarker for a disruption of a BBB can be used to distinguish a subject displaying severe HARM from a subject not displaying HARM. In some cases, a biomarker for a disruption of a BBB can be used to distinguish a subjects displaying mild, intermediate, severe HARM or no HARM from each other.

In some cases, a biomarker can be present in a biological sample obtained or derived from a subject. A biological sample may be blood or any excretory liquid. Non-limiting examples of the biological sample may include saliva, blood, serum, cerebrospinal fluid, semen, feces, plasma, urine, a suspension of cells, or a suspension of cells and viruses. A biological sample may contain whole cells, lysed cells, plasma, red blood cells, skin cells, non-nucleic acids (e.g. proteins), nucleic acids (e.g. DNA, RNA, maternal DNA, maternal RNA), circulating nucleic acids (e.g. cell-free nucleic acids, cell-free DNA/cfDNA, cell-free RNA/cfRNA), circulating tumor DNA/ctDNA, cell-free fetal DNA/cffDNA). In some instances, a sample can contain cell-free nucleic acids. As used herein, the term “cell-free” can refer to the condition of the nucleic acid as it appeared in the body before a sample can be obtained from the body. For example, circulating cell-free nucleic acids in a sample may have originated as cell-free nucleic acids circulating in the bloodstream of the human body. In contrast, nucleic acids that can be extracted from a solid tissue, such as a biopsy, are generally not considered to be “cell-free.”

The cell-free nucleic acids or epigenetic marker discussed above can be specific to one or more tissues, including brain, lung, liver, heart, spleen, pancreas, small intestine, large intestine, skeletal muscle, smooth muscle, skin, bones, adipose tissues, hairs, thyroid, trachea, gall bladder, kidney, ureter, bladder, aorta, vein, esophagus, diaphragm, stomach, rectum, adrenal glands, bronchi, ears, eyes, retina, genitals, hypothalamus, larynx, nose, tongue, spinal cord, or ureters, uterus, ovary, testis, and/or any combination thereof.

The cell-free nucleic acids or biomarkers discussed above can be specific to one or more types of cells, including trichocytes, keratinocytes, gonadotropes, corticotropes, thyrotropes, somatotropes, lactotrophs, chromaffin cells, parafollicular cells, glomus cells melanocytes, nevus cells, merkel cells, odontoblasts, cementoblasts corneal keratocytes, retina muller cells, retinal pigment epithelium cells, neurons, glias (e.g., oligodendrocyte astrocytes), ependymocytes, pinealocytes, pneumocytes (e.g., type I pneumocytes, and type II pneumocytes), clara cells, goblet cells, G cells, D cells, Enterochromaffin-like cells, gastric chief cells, parietal cells, foveolar cells, K cells, D cells, I cells, goblet cells, paneth cells, enterocytes, microfold cells, hepatocytes, hepatic stellate cells (e.g., Kupffer cells from mesoderm), cholecystocytes, centroacinar cells, pancreatic stellate cells, pancreatic α cells, pancreatic β cells, pancreatic δ cells, pancreatic F cells, pancreatic cells, thyroid (e.g., follicular cells), parathyroid (e.g., parathyroid chief cells), oxyphil cells, urothelial cells, osteoblasts, osteocytes, chondroblasts, chondrocytes, fibroblasts, fibrocytes, myoblasts, myocytes, myosatellite cells, tendon cells, cardiac muscle cells, lipoblasts, adipocytes, interstitial cells of cajal, angioblasts, endothelial cells, mesangial cells (e.g., intraglomerular mesangial cells and extraglomerular mesangial cells), juxtaglomerular cells, macula densa cells, stromal cells, interstitial cells, telocytes simple epithelial cells, podocytes, kidney proximal tubule brush border cells, sertoli cells, leydig cells, granulosa cells, peg cells, germ cells, spermatozoon ovums, lymphocytes, myeloid cells, endothelial progenitor cells, endothelial stem cells, angioblasts, mesoangioblasts, pericyte mural cells, and/or any combination thereof.

The methods disclosed herein can assess a disruption of a BBB with high specificity and sensitivity. In some case, one of such methods can comprise one or more steps of: (a) determining in an assay a presence of one or more biomarkers in a biological sample obtained from a subject, where the subject can be a subject having blood brain barrier disruption or suspected of having blood brain barrier disruption, and (b) comparing the presence of the biomarkers in the biological sample obtained from the subject to a reference derived from one or more control samples.

In some cases, a ratio of cell-free nucleic acids carrying a biomarker to total cell-free nucleic acids can be determined. In some cases, a ratio of the cell-free nucleic acids carrying a biomarker to the total cell-free nucleic acids in a sample can be in a range from about 0.01 to about 10000. In some aspects, a ratio of cell-free nucleic acids carrying a biomarker to total cell-free nucleic acids in a sample can be at least about 0.0005, 0.5, 1, 1.5, 2, 3, 4, 5, 6, 7, 8, 9, 10, 20, 30, 40, 50, 60, 70, 80, 90, 100, 200, 500, or at least about 1000. In some aspects, a ratio of the total cell-free nucleic acids in a sample to cell-free nucleic acids carrying a biomarker can be at least about 0.0005, 0.5, 1, 1.5, 2, 3, 4, 5, 6, 7, 8, 9, 10, 20, 30, 40, 50, 60, 70, 80, 90, 100, 200, 500, or at least about 1000. In some cases, a presence or absence of a BB disruption can be determined based on a ratio of cell-free nucleic acids carrying a biomarker to the total cell-free nucleic acids in a sample. In other embodiments, a presence or absence of a BBB disruption can be determined based on a presence or level of a biomarker in cell-free nucleic acids.

Any step of the methods herein can be performed using a computer system as described herein. A computer system can comprise a memory that stores executable instructions and a processor to execute the executable instructions to perform any step of the methods herein. In some cases, one or more of the assessing steps herein can be performed using a computer system.

Any conventional DNA or RNA detection methods can be used for measuring the cell-free nucleic acids. Measuring cell-free nucleic acids can comprise detection of presence, level, amount, and/or concentration of the cell-free nucleic acids. In some cases, any means for detecting low copy number nucleic acids can be used to detect the nucleic acids. Methods for detecting and quantifying low copy number nucleic acids include analytic biochemical methods such as electrophoresis, capillary electrophoresis, high performance liquid chromatography (HPLC), thin layer chromatography (TLC), hyperdiffusion chromatography, mass spectroscopy, spectrophotometry, electrophoresis (e.g., gel electrophoresis), and the like can be utilized. Measuring the level of cell-free nucleic acids can be performed using a polymerase chain reaction (PCR), e.g., any PCR technology described in the disclosure. In some cases, the level of cell-free nucleic acids can be measured by quantitative PCR (e.g., quantitative real-time PCR).

The level of cell-free nucleic acids can be measured by detecting the level of human leukocyte antigen (HLA) locus, mitochondrial DNA, mitochondrial RNA (e.g., mitochondrial mRNA), Y chromosomal genes blood group antigen genes like RHD (cluster of differentiation 240D (CD240D)), ribonuclease P RNA component Hi, Alu J element, endogenous retrovirus group 3, glyceraldehyde 3-phosphate dehydrogenase, N-acetylglucosamine kinase, alcohol dehydrogenase, beta-globin, a member of the albumin family, telomerase reverse transcriptase (TERT), or any combination thereof. Detection of the level of these markers can include the detection the level of the gene (or a fragment thereof), or transcripts, e.g., mRNA (or a fragment thereof) of the markers. In some cases, such a marker can be TERT.

Measuring alevel of cell-free nucleic acids can be performed using a probe. Similarly, measuring the level of cell-free nuclei acids carrying one or more epigenetic markers can be performed using a probe. A probe can bind (e.g., directly or indirectly) to at least one of the cell-free nucleic acids, or at least one of the cell-free nucleic acids carrying one or more epigenetic markers. In some cases, a probe can be labeled. Such probes and labels are disclosed herein. In some cases, a probe can be a polynucleotide. For example the polynucleotide can hybridize with at least one of the cell-free nucleic acids in the sample. In some embodiments, a polynucleotide can be double stranded or single stranded.

When measuring a level of cell-free nucleic acids in a sample, a polynucleotide can be added into the sample as a control (e.g. Exogenous polynucleotide). The level of the exogenous polynucleotide can be indicative of loss or bias during nucleic acid manipulation steps (e.g., isolation, purification or concentration). For example, when isolating or purifying nucleic acid from a sample, the isolating or purification efficiency can be determined by comparing the level of the polynucleotide before and after the isolation or purification step. In some cases, such polynucleotide can be one of a nucleic acid in the sample (e.g., an endogenous polynucleotide). In some cases, such polynucleotide does not exist in the sample, e.g., an exogenous polynucleotide. An exogenous polynucleotide can be synthetic or from another species different from the subject being tested. In some case, an exogenous polynucleotide can be a fluorescence protein (e.g., green fluorescent protein (GFP)) or a fragment thereof. For example, an exogenous polynucleotide can be a fragment of a DNA fragment (e.g., a 605 bp fragment) originating from the GFP-encoding portion of the pontellina plumata genome.

In some embodiments, after measuring a level of cell-free nucleic acids in a sample obtained from a subject, a level of cell-free nucleic acids in a sample can be compared to a reference. A reference can be a level of cell-free nucleic acids in a reference sample from any reference subject described in this disclosure, e.g., a healthy subject, stroke subject or a stroke mimic subject.

Measuring a level of cell-free nucleic acids can be performed by measuring a level of one or more markers (one or more genes or fragments thereof) whose level can be indicative of the level of cell-free nucleic acids in the sample. In some cases, such markers can be present in a subject displaying a disruption of a BBB at a higher level compared to a subject that does not display a disruption of a BBB. In some cases, such markers can be present in a subject displaying a disruption of a BBB at a lower level compared to a subject that does not display a disruption of a BBB. In some cases, a subject that displays a disruption of a BBB also displays HARM as determined by MRI upon administration of a contrast agent. In some cases, a subject that displays a disruption of a BBB does not display HARM. In some cases, a level of one or more biomarkers can be the same in a subject that displays a disruption of a BBB as in a subject that displays HARM. In some cases, a level of one or more biomarkers can be different in a subject that displays a disruption of a BBB than in a subject that displays HARM.

In some exemplary embodiments, a level of a biomarker in a subject that displays mild or intermediate HARM can be the same as a level of the biomarker in a subject who does not display HARM. In this case, a subject who does not display HARM, a subject who displays mild HARM, and a subject who displays intermediate HARM can be grouped into a single phenotype, which can be distinguished from a subject who has severe HARM. In some cases, a level of one or more biomarkers in a subject who has severe HARM can be different than a level of the one or more biomarkers in subjects who does not display HARM, who display mild HARM, and who display intermediate HARM.

An assay can be performed to assess a level or presence of a biomarker, which can be compared with a reference. In some cases, a single biomarker can be used in the assay. In some cases, a group of biomarkers can be used. In some cases, a group of biomarkers can comprise any number of biomarkers. For example, the group of biomarkers can comprise at least about 1, 2, 3, 4, 5, 6, 7, 8, 9, 10, 11, 12, 13, 14, 15, 16, 17, 18, 19, 20, 25, 30, 40, 50, 60, 70, 80, 90, 100, 200, 400, 800, or 1000 biomarkers. In some cases, the group of biomarkers can comprise about 1, 2, 3, 4, 5, 6, 7, 8, 9, 10, 11, 12, 13, 14, 15, 16, 17, 18, 19, 20 biomarkers. A group of biomarkers can be used to detect a disruption of a BBB in a subject. In some cases, a disruption of a BBB can be detected in a subject if a level of the biomarker can be increased compared to a reference. For example, a disruption can be detected in a subject if a level of a biomarker such as cell-free nucleic acids can be increased by at least about 1%, 5%, 7%, 10%, 20%, 40%, 60%, 80%, 1 fold, 2 fold, 3 fold, 4 fold, 5 fold, 10 fold, or 20 fold compared to a reference. Alternatively, a disruption can be detected in a subject if a level of biomarker can be decreased compared to a reference. For example, a disruption can be detected in a subject if a level of a biomarker such as cell-free nucleic acids can be decreased by at least about 1%, 5%, 7%, 10%, 20%, 40%, 60%, 80%, 1 fold, 2 fold, 3 fold, 4 fold, 5 fold, 10 fold, or 20 fold compared to a reference.

A sample can be obtained from a subject prior to the subject exhibiting a hemorrhagic transformation. In some cases, a sample can be obtained from a subject after the subject exhibiting a hemorrhagic transformation. In some cases, a sample can be obtained from a subject after the subject exhibits a symptom of a disruption of a BBB. For example, a sample can be obtained from a subject at least about 0.5, 1, 1.5, 2, 2.5, 3, 3.5, 4, 4.5, 5, 5.5, 6, 6.5, 7, 7.5, 8, 10, 12, 15, 20, 24, 50, 72, 96, or 120 hours from the onset of a symptom of a BBB disruption or a hemorrhagic transformation. A sample can be obtained from a subject at least about 0.5, 1, 1.5, 2, 2.5, 3, 3.5, 4, 4.5, 5, 5.5, 6, 6.5, 7, 7.5, 8, 10, 12, 15, 20, 24, 50, 72, 96, or 120 hours from the onset of a symptom of a stroke. A sample can be obtained from a subject at least about 0.5, 1, 1.5, 2, 2.5, 3, 3.5, 4, 4.5, 5, 5.5, 6, 6.5, 7, 7.5, 8, 10, 12, 15, 20, 24, 50, 72, 96, or 120 hours prior to the onset of a symptom of a stroke, hemorrhagic transformation or a BBB disruption.

Assessing a disruption of a BBB in a subject can comprise one or more of the following: a) determining whether the subject can be at risk or has previously displayed a disruption of a BBB; b) assessing the risk of the subject for having a disruption of a BBB; c) assessing a risk of the subject developing a condition associated with a disruption of a BBB (e.g. a stroke); d) predicting the severity of the disruption of the BBB; e) assessing the activation of innate immune system (e.g., assessing the neutrophil count in the subject); f) assessing an injury (e.g., myocardial infarction), and g) assessing a risk of a stroke. One or more assessments can be performed based on a level of a biomarker. For example, neutrophil count can be determined based on a level of a biomarker such as cell-free nucleic acids in the sample. In other embodiments, a method disclosed herein can be used in conjunction with a second method to make an assessment.

The methods disclosed herein can comprise determining a risk of developing an ischemic stroke symptom onset in a subject. In some cases, a time of developing an ischemic stroke symptom can be determined by correlating the level of a biomarker in a sample with the time of onset of a disruption of a BBB.

The provided methods can increase the accuracy of diagnosing a blood brain barrier disruption. The provided methods herein can provide increased specificity and specificity. Several prior studies have looked to identify circulating plasma proteins which can be associated with hemorrhagic transformation; for the most part, these studies have targeted proteins which can be either involved in the breakdown of the BBB or released as a result. Such proteins include matrix metalloproteases, tight junctional proteins, and proteins which can be largely specific to the cells of the CNS. The most promising of these proteins has proven to be s100b, a calcium binding protein which can be expressed predominantly by the glial cells of the CNS. While multiple reports have agreed that circulating s100b can be elevated early in ischemic stroke patients who later undergo hemorrhagic transformation, studies targeting s100b have not demonstrated levels of diagnostic robustness which suggest it could be a clinically useful biomarker. In the largest clinical study which evaluated the ability of s100b levels to identify patients at risk for hemorrhagic transformation, s100b was only able to identify such patients with 92.9% sensitivity and 48.1% specificity. The panel of biomarkers which are identified herein outperform the majority of protein based biomarkers which have been previously evaluated for their ability to predict post-stroke BBB disruption using different criteria to classify disruption of the blood brain barrier.

One previous study has used RNA expression profiling to identify potential transcriptional biomarkers which could be used to predict hemorrhagic transformation. In this study, a panel of six gene products was identified whose expression levels showed the ability to discriminate between 11 ischemic stroke patients with hemorrhagic transformation and 33 without hemorrhagic transformation with 72.7% sensitivity and 93.9% specificity in a discovery cohort, and between 5 ischemic stroke patients with hemorrhagic transformation and 47 without hemorrhagic transformation with 80% sensitivity and 72% specificity in a separate validation cohort. The biomarker panel identify herein outperform this previously identified panel in terms of identifying post-stroke blood brain barrier disruption

Provided herein include methods for identifying biomarkers of BBB disruption. The methods disclosed herein can comprise measuring a profile of polynucleotides in a sample from a subject displaying mild or no HARM, and measuring a profile of polynucleotides in a second sample from a subject displaying severe HARM. A group of biomarkers can be identified by comparing the profile of polynucleotides in the first sample to a polynucleotide reference profile. For example, a group of biomarkers can include genes whose expression levels can be up-regulated or down-regulated in the first sample relative to the second sample.

A sample can be fresh or frozen, and/or can be treated, e.g. with heparin, citrate, or EDTA. A sample can also include sections of tissues such as frozen sections taken for histological purposes. In some cases, a sample can be a sample derived from a subject with a BBB disruption or having a risk of BBB disruption. In some cases, a sample can be a sample derived from a subject with a BBB disruption. For example, a sample can be derived from a subject with a BBB disruption within a range of about 0.5 hours to about 120 hours of presentation of at least one symptom of a BBB disruption. In a particular example, a sample can derived from a subject displaying BBB disruption within about 0.5, 1, 1.5, 2, 2.5, 3, 3.5, 4, 4.5, 5, 5.5, 6, 6.5, 7, 7.5, 8, 10, 12, 15, 20, 24, 50, 72, 96, 120, 150, or 200 hours of at least one symptom.

In some cases, a sample can be a biological fluid. When a sample is a biological fluid, the volume of the fluidic sample can be greater than 1 mL (milliliter). In some cases, the volume of the fluidic sample can be within a range of at least about 1.0 mL to about 15 mL. For example, the volume of the sample can be about 1.0 mL, 1.1 mL, 1.2 mL, 1.4 mL, 1.6 mL, 1.8 mL, 1.9 mL, 2 mL, 3 mL, 4 mL, 5 mL, 6 mL, 7 mL, 8 mL, 9 mL, or 10 mL. Alternatively, in some cases, the volume of the fluidic sample can be no greater than 1 mL. For example, the volume of the sample can be less than about 0.00001 mL, 0.0001 mL, 0.001 mL, 0.01 mL, 0.1 mL, 0.2 mL, 0.4 mL, 0.6 mL, 0.8 mL, 1 mL.

A sample disclosed herein can be blood. For example, a sample can be peripheral blood. In some cases, a sample can be a fraction of blood. In one example, a sample can be serum. In another example, a sample can be plasma. In another example, a sample can include one or more cells circulating in blood. Such cells can include red blood cells (e.g., erythrocytes), white blood cells (e.g., leukocytes, including, neutrophils, eosinophils, basophils, lymphocyte, and monocytes (e.g., peripheral blood mononuclear cell)), platelets (e.g., thrombocytes), circulating tumor cells, or any type of cells circulating in peripheral blood and combinations thereof.

A sample can be derived from a subject. In some cases, a subject can be a human, e.g. a human patient. In some cases, a subject can be a non-human animal, including a mammal such as a domestic pet (e.g., a dog, or a cat) or a primate. A sample can contain one or more polypeptide or protein biomarkers, or a polynucleotide biomarker disclosed herein (e.g., mRNA). A subject can be suspected of having a condition (e.g., a disease).

In some cases, a disruption of a BBB can lead to a stroke. In some cases, a disruption of a BBB may not lead to a stroke. In some cases, a stroke can lead to a disruption of a BBB. In some cases, a stroke may not lead to a disruption of a BBB. Stroke can refer to a medical condition that can occur when the blood supply to part of the brain may be interrupted or severely reduced, depriving brain tissue of oxygen and nutrients. Within minutes, brain cells can begin to die. Stroke can include ischemic stroke, hemorrhagic stroke and transient ischemic attack (TIA). Ischemic stroke can occur when there can be a decrease or loss of blood flow to an area of the brain resulting in tissue damage or destruction. Hemorrhagic stroke can occur when a blood vessel located in the brain is ruptured leading to the leakage and accumulation of blood directly in the brain tissue. Transient ischemic attack or mini stroke, can occur when a blood vessel is temporarily blocked. Ischemic stroke can include thrombotic, embolic, lacunar and hypoperfusion types of strokes.

An ischemic stroke subject can refer to a subject with an ischemic stroke or having a risk of having an ischemic stroke. In some cases, an ischemic stroke subject can be a subject that has had ischemic stroke within 24 hours. In a particular example, an ischemic stroke subject can be a subject that has had an ischemic stroke within 4.5 hours. A non-ischemic stroke subject can be a subject who has not had an ischemic stroke. In some cases, a non-ischemic stroke subject can be a subject who has not had an ischemic stroke and has no risk of having an ischemic stroke.

A subject with stroke (e.g., ischemic stroke) can have one or more stroke symptoms. Stroke symptoms can be present at the onset of any type of stroke (e.g., ischemic stroke or hemorrhagic stroke). Stroke symptoms can be present before or after the onset of any type of stroke. Stroke symptoms can include those symptoms recognized by the National Stroke Association, which include: (a) sudden numbness or weakness of the face, arm or leg—especially on one side of the body; (b) sudden confusion, trouble speaking or understanding; (c) sudden trouble seeing in one or both eyes; (d) sudden trouble walking, dizziness, loss of balance or coordination, and (e) sudden severe headache with no known cause.

A non-ischemic stroke subject can have stroke-mimicking symptoms. Stroke-mimicking symptoms can include pain, headache, aphasia, apraxia, agnosia, amnesia, stupor, confusion, vertigo, coma, delirium, dementia, seizure, migraine insomnia, hypersomnia, sleep apnea, tremor, dyskinesia, paralysis, visual disturbances, diplopia, paresthesia, dysarthria, hemiplegia, hemianesthesia, and hemianopia. When a stroke-mimicking symptom is present in a subject that has not suffered a stroke, the symptoms can be referred to as “stroke mimics”. Conditions within the differential diagnosis of stroke include brain tumor (e.g., primary and metastatic disease), aneurysm, electrocution, burns, infections (e.g., meningitis), cerebral hypoxia, head injury (e.g. concussion), traumatic brain injury, stress, dehydration, nerve palsy (e.g., cranial or peripheral), hypoglycemia, migraine, multiple sclerosis, peripheral vascular disease, peripheral neuropathy, seizure (e.g., grand mal seizure), subdural hematoma, syncope, and transient unilateral weakness. Biomarkers of ischemic stroke disclosed herein can be those that can distinguish acute ischemic stroke from these stroke-mimicking conditions. In some cases, the biomarkers disclosed herein can identify a stroke mimicking condition disclosed herein. In some cases, the biomarkers disclosed herein can identify a non-stroke condition disclosed herein.

The methods, devices, and kits herein can be used to assess a condition. A condition can be a disease or a risk of a disease in a subject. For example, the methods can comprise measuring the expression of a group of biomarkers in a sample from a subject, and assessing a disease or a risk of a disease in a subject based on the expression. In some cases, a condition can be a risk factor for strokes or BBB disruption, e.g., high blood pressure, atrial fibrillation, high cholesterol, diabetes, atherosclerosis, circulation problems, tobacco use, alcohol use, physical inactivity, obesity, age, gender, race, family history, previous stroke, previous transient ischemic attack (TIA), fibromuscular dysplasia, patent foramen ovale, or any combination thereof. If one or more risk factors are known in a subject, the risk factors can be used, e.g., in combination with the expression of a group of biomarkers, to assess BBB disruption and or a risk of ischemic stroke in the subject.

In some instances, a disruption of a BBB can result in a condition associated with the disruption. A condition can be a disease. A disease can be BBB disruption or a BBB disruption associated disease. A disease can be ischemic stroke. In some cases, a disease can be Alzheimer's disease or Parkinson's disease. In some cases, a disease can be an autoimmune disease such as acute disseminated encephalomyelitis (ADEM), acute necrotizing hemorrhagic leukoencephalitis, Addison's disease, agammaglobulinemia, allergic asthma, allergic rhinitis, alopecia areata, amyloidosis, ankylosing spondylitis, anti-GBM/anti-TBM nephritis, antiphospholipid syndrome (APS), autoimmune aplastic anemia, autoimmune dysautonomia, autoimmune hepatitis, autoimmune hyperlipidemia, autoimmune immunodeficiency, autoimmune inner ear disease (AIED), autoimmune myocarditis, autoimmune pancreatitis, autoimmune retinopathy, autoimmune thrombocytopenic purpura (ATP), autoimmune thyroid disease, axonal & neuronal neuropathies, Balo disease, Behcet's disease, bullous pemphigoid, cardiomyopathy, Castlemen disease, celiac sprue (non-tropical), Chagas disease, chronic fatigue syndrome, chronic inflammatory demyelinating polyneuropathy (CIDP), chronic recurrent multifocal ostomyelitis (CRMO), Churg-Strauss syndrome, cicatricial pemphigoid/benign mucosal pemphigoid, Crohn's disease, Cogan's syndrome, cold agglutinin disease, congenital heart block, coxsackie myocarditis, CREST disease, essential mixed cryoglobulinemia, demyelinating neuropathies, dermatomyositis, Devic's disease (neuromyelitis optica), discoid lupus, Dressler's syndrome, endometriosis, eosinophillic fasciitis, erythema nodosum, experimental allergic encephalomyelitis, Evan's syndrome, fibromyalgia, fibrosing alveolitis, giant cell arteritis (temporal arteritis), glomerulonephritis, Goodpasture's syndrome, Grave's disease, Guillain-Barre syndrome, Hashimoto's encephalitis, Hashimoto's thyroiditis, hemolytic anemia, Henock-Schoniein purpura, herpes gestationis, hypogammaglobulinemia, idiopathic thrombocytopenic purpura (ITP), IgA nephropathy, immunoregulatory lipoproteins, inclusion body myositis, insulin-dependent diabetes (type 1), interstitial cystitis, juvenile arthritis, juvenile diabetes, Kawasaki syndrome, Lambert-Eaton syndrome, leukocytoclastic vasculitis, lichen planus, lichen sclerosus, ligneous conjunctivitis, linear IgA disease (LAD), Lupus (SLE), Lyme disease, Meniere's disease, microscopic polyangitis, mixed connective tissue disease (MCTD), Mooren's ulcer, Mucha-Habermann disease, multiple sclerosis, myasthenia gravis, myositis, narcolepsy, neuromyelitis optica (Devic's), neutropenia, ocular cicatricial pemphigoid, optic neuritis, palindromic rheumatism, PANDAS (Pediatric Autoimmune Neuropsychiatric Disorders Associated with Streptococcus), paraneoplastic cerebellar degeneration, paroxysmal nocturnal hemoglobinuria (PNH), Parry Romberg syndrome, Parsonnage-Turner syndrome, pars plantis (peripheral uveitis), pemphigus, peripheral neuropathy, perivenous encephalomyelitis, pernicious anemia, POEMS syndrome, polyarteritis nodosa, type I, II & III autoimmune polyglandular syndromes, polymyalgia rheumatic, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasis, Raynaud's phenomena, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, Slogren's syndrome, sperm and testicular autoimmunity, stiff person syndrome, subacute bacterial endocarditis (SBE), sympathetic ophthalmia, Takayasu's arteritis, temporal arteritis/giant cell arteries, thrombocytopenic purpura (TPP), Tolosa-Hunt syndrome, transverse myelitis, ulcerative colitis, undifferentiated connective tissue disease (UCTD), uveitis, vasculitis, vesiculobullous dermatosis, vitiligo or Wegener's granulomatosis or, chronic active hepatitis, primary biliary cirrhosis, cadilated cardiomyopathy, myocarditis, autoimmune polyendocrine syndrome type I (APS-I), cystic fibrosis vasculitides, acquired hypoparathyroidism, coronary artery disease, pemphigus foliaceus, pemphigus vulgaris, Rasmussen encephalitis, autoimmune gastritis, insulin hypoglycemic syndrome (Hirata disease), Type B insulin resistance, acanthosis, systemic lupus erythematosus (SLE), pernicious anemia, treatment-resistant Lyme arthritis, polyneuropathy, demyelinating diseases, atopic dermatitis, autoimmune hypothyroidism, vitiligo, thyroid associated ophthalmopathy, autoimmune coeliac disease, ACTH deficiency, dermatomyositis, Sjogren syndrome, systemic sclerosis, progressive systemic sclerosis, morphea, primary antiphospholipid syndrome, chronic idiopathic urticaria, connective tissue syndromes, necrotizing and crescentic glomerulonephritis (NCGN), systemic vasculitis, Raynaud syndrome, chronic liver disease, visceral leishmaniasis, autoimmune C1 deficiency, membrane proliferative glomerulonephritis (MPGN), prolonged coagulation time, immunodeficiency, atherosclerosis, neuronopathy, paraneoplastic pemphigus, paraneoplastic stiff man syndrome, paraneoplastic encephalomyelitis, subacute autonomic neuropathy, cancer-associated retinopathy, paraneoplastic opsoclonus myoclonus ataxia, lower motor neuron syndrome and Lambert-Eaton myasthenic syndrome.

In some cases, a disease can be a cancer such as Acute lymphoblastic leukemia, Acute myeloid leukemia, Adrenocortical carcinoma, AIDS-related cancers, AIDS-related lymphoma, Anal cancer, Appendix cancer, Astrocytoma, childhood cerebellar or cerebral, Basal cell carcinoma, Bile duct cancer, extrahepatic, Bladder cancer, Bone cancer, Osteosarcoma/Malignant fibrous histiocytoma, Brainstem glioma, Brain tumor, Brain tumor, cerebellar astrocytoma, Brain tumor, cerebral astrocytoma/malignant glioma, Brain tumor, ependymoma, Brain tumor, medulloblastoma, Brain tumor, supratentorial primitive neuroectodermal tumors, Brain tumor, visual pathway and hypothalamic glioma, Breast cancer, Bronchial adenomas/carcinoids, Burkitt lymphoma, Carcinoid tumor, childhood, Carcinoid tumor, gastrointestinal, Carcinoma of unknown primary, Central nervous system lymphoma, primary, Cerebellar astrocytoma, childhood, Cerebral astrocytoma/Malignant glioma, childhood, Cervical cancer, Childhood cancers, Chronic lymphocytic leukemia, Chronic myelogenous leukemia, Chronic myeloproliferative disorders, Colon Cancer, Cutaneous T-cell lymphoma, Desmoplastic small round cell tumor, Endometrial cancer, Ependymoma, Esophageal cancer, Ewing's sarcoma in the Ewing family of tumors, Extracranial germ cell tumor, Childhood, Extragonadal Germ cell tumor, Extrahepatic bile duct cancer, Eye Cancer, Intraocular melanoma, Eye Cancer, Retinoblastoma, Gallbladder cancer, Gastric (Stomach) cancer, Gastrointestinal Carcinoid Tumor, Gastrointestinal stromal tumor (GIST), Germ cell tumor: extracranial, extragonadal, or ovarian, Gestational trophoblastic tumor, Glioma of the brain stem, Glioma, Childhood Cerebral Astrocytoma, Glioma, Childhood Visual Pathway and Hypothalamic, Gastric carcinoid, Hairy cell leukemia, Head and neck cancer, Heart cancer, Hepatocellular (liver) cancer, Hodgkin lymphoma, Hypopharyngeal cancer, Hypothalamic and visual pathway glioma, childhood, Intraocular Melanoma, Islet Cell Carcinoma (Endocrine Pancreas), Kaposi sarcoma, Kidney cancer (renal cell cancer), Laryngeal Cancer, Leukemias, Leukemia, acute lymphoblastic (also called acute lymphocytic leukemia), Leukemia, acute myeloid (also called acute myelogenous leukemia), Leukemia, chronic lymphocytic (also called chronic lymphocytic leukemia), Leukemia, chronic myelogenous (also called chronic myeloid leukemia), Leukemia, hairy cell, Lip and Oral Cavity Cancer, Liver Cancer (Primary), Lung Cancer, Non-Small Cell, Lung Cancer, Small Cell, Lymphomas, Lymphoma, AIDS-related, Lymphoma, Burkitt, Lymphoma, cutaneous T-Cell, Lymphoma, Hodgkin, Lymphomas, Non-Hodgkin (an old classification of all lymphomas except Hodgkin's), Lymphoma, Primary Central Nervous System, Marcus Whittle, Deadly Disease, Macroglobulinemia, Waldenström, Malignant Fibrous Histiocytoma of Bone/Osteosarcoma, Medulloblastoma, Childhood, Melanoma, Melanoma, Intraocular (Eye), Merkel Cell Carcinoma, Mesothelioma, Adult Malignant, Mesothelioma, Childhood, Metastatic Squamous Neck Cancer with Occult Primary, Mouth Cancer, Multiple Endocrine Neoplasia Syndrome, Childhood, Multiple Myeloma/Plasma Cell Neoplasm, Mycosis Fungoides, Myelodysplastic Syndromes, Myelodysplastic/Myeloproliferative Diseases, Myelogenous Leukemia, Chronic, Myeloid Leukemia, Adult Acute, Myeloid Leukemia, Childhood Acute, Myeloma, Multiple (Cancer of the Bone-Marrow), Myeloproliferative Disorders, Chronic, Nasal cavity and paranasal sinus cancer, Nasopharyngeal carcinoma, Neuroblastoma, Non-Hodgkin lymphoma, Non-small cell lung cancer, Oral Cancer, Oropharyngeal cancer, Osteosarcoma/malignant fibrous histiocytoma of bone, Ovarian cancer, Ovarian epithelial cancer (Surface epithelial-stromal tumor), Ovarian germ cell tumor, Ovarian low malignant potential tumor, Pancreatic cancer, Pancreatic cancer, islet cell, Paranasal sinus and nasal cavity cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, childhood, Pituitary adenoma, Plasma cell neoplasia/Multiple myeloma, Pleuropulmonary blastoma, Primary central nervous system lymphoma, Prostate cancer, Rectal cancer, Renal cell carcinoma (kidney cancer), Renal pelvis and ureter, transitional cell cancer, Retinoblastoma, Rhabdomyosarcoma, childhood, Salivary gland cancer, Sarcoma, Ewing family of tumors, Sarcoma, Kaposi, Sarcoma, soft tissue, Sarcoma, uterine, Sézary syndrome, Skin cancer (nonmelanoma), Skin cancer (melanoma), Skin carcinoma, Merkel cell, Small cell lung cancer, Small intestine cancer, Soft tissue sarcoma, Squamous cell carcinoma—see Skin cancer (nonmelanoma), Squamous neck cancer with occult primary, metastatic, Stomach cancer, Supratentorial primitive neuroectodermal tumor, childhood, T-Cell lymphoma, cutaneous—see Mycosis Fungoides and Sézary syndrome, Testicular cancer, Throat cancer, Thymoma, childhood, Thymoma and Thymic carcinoma, Thyroid cancer, Thyroid cancer, childhood, Transitional cell cancer of the renal pelvis and ureter, Trophoblastic tumor, gestational, Unknown primary site, carcinoma of, adult, Unknown primary site, cancer of, childhood, Ureter and renal pelvis, transitional cell cancer, Urethral cancer, Uterine cancer, endometrial, Uterine sarcoma, Vaginal cancer, Visual pathway and hypothalamic glioma, childhood, Vulvar cancer, Waldenström macroglobulinemia, Wilms tumor (kidney cancer), childhood.

In some cases, a disease can be inflammatory disease, infectious disease, cardiovascular disease and metabolic disease. Specific infectious diseases include, but is not limited to AIDS, anthrax, botulism, brucellosis, chancroid, chlamydial infection, cholera, coccidioidomycosis, cryptosporidiosis, cyclosporiasis, dipheheria, ehrlichiosis, arboviral encephalitis, enterohemorrhagic Escherichia coli, giardiasis, gonorrhea, dengue fever, haemophilus influenza, Hansen's disease (Leprosy), hantavirus pulmonary syndrome, hemolytic uremic syndrome, hepatitis A, hepatitis B, hepatitis C, human immunodeficiency virus, legionellosis, listeriosis, lyme disease, malaria, measles. Meningococcal disease, mumps, pertussis (whooping cough), plague, paralytic poliomyelitis, psittacosis, Q fever, rabies, rocky mountain spotted fever, rubella, congenital rubella syndrome (SARS), shigellosis, smallpox, streptococcal disease (invasive group A), streptococcal toxic shock syndrome, Streptococcus pneumonia, syphilis, tetanus, toxic shock syndrome, trichinosis, tuberculosis, tularemia, typhoid fever, vancomycin intermediate resistant staphylocossus aureus, varicella, yellow fever, variant Creutzfeldt-Jakob disease (vCJD), Eblola hemorrhagic fever, Echinococcosis, Hendra virus infection, human monkeypox, influenza A, H5N1, lassa fever, Margurg hemorrhagic fever, Nipah virus, O'nyong fever, Rift valley fever, Venezuelan equine encephalitis and West Nile virus.

In some embodiments, the methods, device and kits described herein can detect one or more of the diseases disclosed herein. In some embodiments, one or more of the biomarkers disclosed herein can be used to assess one or more disease disclosed herein. In some embodiments, one or more of the biomarkers disclosed herein can be used to detect one or more diseases disclosed herein.

The group of biomarkers disclosed herein can comprise one or more of an anthrax toxin receptor, a serine/threonine-protein kinase, a chemokine, a pyruvate dehydrogenase lipoamide kinase, a cluster of differentiation family member, myelin and lymphocyte protein (MAL), an inhibitor of Ras-ERK pathway, a member of inhibitor of DNA binding family, a lysosomal cysteine proteinase, a motor protein, and a receptor for pigment epithelium-derived factor. A group of biomarkers can be involved in various pathways. For example, a biomarker can be involved in chemotaxis, response to cyclic AMP, an RNA catabolic process, ganulocyte-mediated immunity, response to a lipopolysaccharide, toll-like receptor signaling, locomotory behavior, response to wound healing, or immunity and defense. In some cases, a biomarker can be involved in at least 1, 2, 3, 4, 5, 6, 7, 8, or 9 processes such as chemotaxis, response to cyclic AMP, an RNA catabolic process, ganulocyte-mediated immunity, response to a lipopolysaccharide, toll-like receptor signaling, locomotory behavior, response to wound healing, or immunity and defense

The group of biomarkers herein can comprise any number of biomarkers. For example, the group of biomarkers can comprise at least about 1, 2, 3, 4, 5, 6, 7, 8, 9, 10, 11, 12, 13, 14, 15, 16, 17, 18, 19, 20, 25, 30, 40, 50, 60, 70, 80, 90, 100, 200, 400, 800, or 1000 biomarkers. In some cases, the group of biomarkers can comprise about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 biomarkers. In some cases, a group of biomarker can comprise about 1, 2, 3, 4, 5, 6, 7, 8, 9, 10, 11, 12, 13, 14, 15, 16, 17, 18, 19, 20, 21, 22, 23, 24, or 25 of a biomarker recited in Table 1. In some cases, a group of biomarker can comprise about 1, 2, 3, 4, 5, 6, 7, 8, 9, 10, 11, 12, 13, 14, 15, 16, 17, 18, 19, 20, 21, 22, 23, 24, or 25 of LAIR2, IL-8, CXCL5, RBP7, CCDC149, LY96, DDIT4, HPSE, E2F3, ADAM15, ACN9, TMEM176B, BIRC2, EMR2, DUSP1, HSPA1B, RNASE2, IDI1, SCOC, FAM65A, CD14, F2RL1, PCMTD1, SMEK2, or SDPR. In some cases, the group of biomarkers can comprise about 1, 2, 3, 4, or 5 of RBP7, CCDC149, DDIT4, E2F3, or ADAM15.

The amino acid and corresponding nucleic acid sequences of exemplary biomarkers are known in the art and can be found in publicly available publications and databases. Exemplary sequences are set forth in Table 1 in the form of GenBank accession numbers.

TABLE 1
Exemplary biomarkers and accession numbers

| | Accession No. | Accession No. |
|---|---|---|
| Gene name | (mRNA) | (protein) |
| ADAM metallopeptidase | NM_001261464.1 | NP 001248393.1 |
| domain 15 (ADAM15) | | |
| Adhesion G protein- | NM_001271052.1 | NP 001257981.1 |
| coupled receptor E2 | | |
| (EMR2) | | |
| Aggrecan (transcript | NM_001135 | AAH36445 |
| variant 1) | | |
| Amyloid precursor | NM_000484 | BAA22264 |
| protein (APP) (transcript | | |
| variant 1) | | |
| Arginase, liver (ARG 1) | NM_000045.2 | NP 000036.2 |
| Baculoviral IAP repeat | NM_001166.4 | NP 001157.1 |
| containing 2 (BIRC2) | | |
| B-cell activating factor | AF116456 | AAD25356 |
| (BAFF) | | |
| Carbonic anhydrase IV | NM_000717.3 | NP 000708.1 |
| (CA4) | | |
| CD14 molecule (CD14) | NM_000591.3 | NP 000582.1 |
| CD27 | NM_001242.4 | NP 001233.1 |
| CD30 (isoform 1 | NM_001243.4 | CAC16652 |
| precursor) | | |
| CD40 (isoform 1) | NM_001250.5 | AAH64518 |
| Chemokine (C—C motif) | NM_001838.2 | NP 001829 |
| receptor 7 (CCR7) | | |
| Coiled-coil domain | NM_001130726.3 | NP 001124198.1 |
| containing 149 | | |
| (CCDC149) | | |
| C—X—C motif chemokine | NM_002994.4 | NP 002985.1 |
| ligand 5 (CXCL5) | | |
| CXCL13 (BLCBCA) | NM_006419.2 | AAH12589.1 |
| DNA damage inducible | NM_019058.3 | NP 061931.1 |
| transcript 4 (DDIT4) | | |
| Dual specificity | NM_004417.3 | NP 004408.1 |
| phosphatase 1 (DUSP1) | | |
| E2F transcription factor 3 | NM_001243076.2 | NP 001230005.1 |
| (E2F3) | | |
| Ephrin-A2 | NM_001405.3 | EAW69517 |
| Epithelial-derived | NM_002994.4 | CAG33709 |
| neutrophil-activating | | |
| peptide 78 (ENA-78) | | |
| F2R like trypsin receptor | NM_005242.5 | NP_005233.3 |
| 1 (F2RL1) | | |
| Family with sequence | NM_001193522.1 | NP 00118045.1 |
| similarity 65, member A | | |
| (FAM65A) | | |
| Fas (isoform 1 precursor) | NM_007987.2 | AAH12479 |
| Galectin-3 | AB006780 | BAA22164 |
| Granulocyte-macrophage | NM_000758.3 | AAA98768 |
| colony-stimulating factor | | |
| (GMCSF) | | |
| GRO-alpha | NM_001511 | AAH11976.1 |
| Heat shock protein family | NM_005346.4 | NP 005337.2 |
| A 1B (HSPA1B) | | |
| Heparanase (HPSE) | NM_001098540.2 | NP 0041092010.1 |
| IFNγ | NM_000619.2 | NP 000610.2 |
| IL-10 | XM_011509506.1 | XP_011507808.1 |
| IL12/IL-23 p70 | NM_002187.2 | NP_002178.2 |
| IL-17 | NM_002190.2 | AAC50341 |
| IL-1α | NM_000575.3 | NP_000566.3 |
| IL-1β | NM_000576.2 | NP_000567.1 |
| IL-2 | NM_000586.3 | AAB46883 |
| IL-31 | XM_011538326.1 | EAW98310 |
| IL-33 (isoform 3) | NM_033439.3 | AAH47085.1 |
| IL-4 (isoform 1) | NM_000589.3 | AAH70123 |
| IL-5 | NM_000879.2 | AAA98620.1 |
| IL-6 | NM_000600.3 | NP_000591.1 |
| IL-8 | NM_000584.3 | NP_000575.1 |
| IQ motif containing | NM_003870.3 | NP_003861.1 |
| GTPase activating | | |
| protein 1 (IQGAP 1) | | |
| Isopentenyl-diphosphate | NM_001317955.1 | NP 001304884.1 |
| delta isomerase 1 (IDI1) | | |
| Leukocyte associated | NM_002288.5 | NP 002279.2 |
| immunoglobulin like | | |
| receptor 2 (LAIR2) | | |
| Lymphocyte antigen 96 | NM_015364.3 | NP 056179.2 |
| (LY96) | | |
| Matrix metallopeptidase | NM_004994.2 | NP 004985.2 |
| 9 (gelatinase B, 92 kDa | | |
| gelatinase, 92 kDa type | | |
| IV collagenase) (MMP9) | | |
| Monocyte chemotactic | NM_005623.2 | CAA71760.1 |
| protein 2 (MCP-2) | | |
| Orosomucoid 1 (ORM 1) | NM_000607.2 | NP 000598.2 |
| Protein-L-isoaspartate | NM_001286782.1 | NM_001273711.1 |
| (D-aspartate) O- | | |
| methyltransferase domain | | |
| containing 1 (PCMTD1) | | |
| Receptor for Advanced | NM_001136.4 | AAH26069 |
| Glycation Endproducts | | |
| (RAGE) (isoform 1 | | |
| precursor) | | |
| Regulated on activation, | NM_002985.2 | EAW80120 |
| normal T cell expressed | | |
| and secreted (RANTES) | | |
| (isoform 1) | | |
| Retinol binding protein 7 | NM_052960.2 | NP 443192.1 |
| (RBP7) | | |
| Ribonuclease A family | NM_002934.2 | NP 002925.1 |
| member 2 (RNASE2) | | |
| S100 calcium binding | NM_005621.1 | NP_005612.1 |
| protein A12 (S100A12) | | |
| Serum deprivation | NM_004657.5 | NP_004648.1 |
| response (SDPR) | | |
| Short coiled-coil protein | NM_001153446.1 | NP_001146918.1 |
| (SCOC) | | |
| SMEK homolog 2, | NM_001122964.2 | NP 001116436.2 |
| suppressor of mek1 | | |
| (SMEK2) | | |
| Succinate dehydrogenase | NM_020186.2 | NP 064571.1 |
| complex assembly factor | | |
| 3 (ACN9) | | |
| Thymus and activation- | NM_002987.2 | EAW82921.1 |
| regulated chemokine | | |
| (TARC) | | |
| TNFR1 | NM_001065.3 | AAA61201 |
| TNFα | NM_000594.3 | NP 000585.2 |
| Transmembrane protein | NM_001101311.1 | NP 001094781.1 |
| 176B (TMEM176B) | | |
| Versican (VCAN) | NM_004385.2 | NP 004376 |
| (CSPG2) | | |

A biomarker can exist in multiple forms, each of which is encompassed herein. For example, variants of a biomarker herein can exist in which a small number, e.g., 1, 2, 3, 4, 5, 6, 7, 8, 9, 10 or more, of nucleotides or amino acid residues are different in relation to the exemplary accession numbers set forth in Table 1. However, these variants are intended to be used in the methods, kits and devices herein. In addition, a biomarker herein can also include the “derivatives” of the biomarker. A “derivative” of a biomarker (or of its encoding nucleic acid molecule) to a modified form of the biomarker. A modified form of a given biomarker can include at least one amino acid substitution, deletion, insertion or combination thereof, where said modified form retains a biological activity of an unmodified form. An amino acid substitution can be considered “conservative” when the substitution results in similar structural or chemical properties (e.g., replacement of leucine with isoleucine). An amino acid substitution can be “non-conservative” in nature where the structure and chemical properties vary (e.g., replacement of arginine with alanine). A modified form of a given biomarker can include chemical modifications, where a modified form retains a biological activity of a given biomarker. Such modifications include, but are not limited to, glycosylation, phosphorylation, acetylation, alkylation, methylation, biotinylation, glutamylation glycylation, isoprenylation, lipoylation, pegylation, phosphopantetheinylation, sulfation, selenation, and C-terminal amidation. Other modifications include those involving other proteins such as ISGylation, SUMOylation, and ubiquitination. In addition, modifications can also include those involved in changing the chemical nature of an amino acid such as deamination and deamidation.

Biomarkers herein can include biomarkers that pertain to other diseases or conditions other than BBB disruption, including stroke or other non-stroke conditions. Non-limiting examples of other biomarkers that can be determined include those related to blood pressure (e.g., A-type natriuretic peptide, C-type antriuretic peptide, urotensin II, vasopressen, calcitonin, angiotensin II, adrenomedullin, and endothenlins), coagulation and hemostasis (e.g., D-dimer, plasmin, b-thromboglobulin, platelet factor 4, fibrinopeptide A, platelet-derived growth factor, prothrombin, P-selectin and thrombin), acute phase response (e.g., C-reactive protein, mannose-binding protein, human neutrophil elastase, inducible nitric oxide synthase, lysophosphatidic acid, malondialdehyde LDL, lipopolysaccharide binding protein) and biomarkers related to inflammation (e.g., interleukins, tumor necrosis factor, myeloperoxidase, soluble intercellular adhesion molecule, vascular cell adhesion molecule, monocyte chemotactic protein-1). Such other biomarkers can assist in gaining a better overall clinical picture of the health of a patient and the potential causes of stroke. Such biomarkers can be selected on the basis of the knowledge of one of ordinary skill in the art. Additional examples of such biomarkers can be found in the art, for example, in U.S. Pat. No. 7,608,406, which is incorporated herein by reference in its entirety.

In some cases, a profile of polynucleotides can comprise an expression pattern of the polynucleotides. For example, an expression pattern of the polynucleotides can be the expression level of the polynucleotides. In another example, an expression pattern of the polynucleotides can be the expression level differences of the polynucleotides compared to a polynucleotides reference profile.

A profile of polynucleotides can be measured by a nucleic acid analysis method. In some cases, a nucleic acid analysis method can be a polymerase chain reaction (PCR). Examples of PCR include amplified fragment length polymorphism PCR, allele-specific PCR, Alu PCR, asymmetric PCR, colony PCR, helicase dependent PCR, hot start PCR, inverse PCR, in situ PCR, intersequence-specific PCR, digital PCR, droplet digital PCR, linear-after-the-exponential-PCR (Late PCR), long PCR, nested PCR, duplex PCR, multiplex PCR, quantitative PCR, or single cell PCR. In a particular example, the nucleic acid analysis method can be quantitative PCR. In some cases, quantitative PCR can be real-time PCR, e.g., real-time quantitative PCR. In real-time quantitative PCR, the accumulation of amplification product can be measured continuously in both standard dilutions of target DNA and samples containing unknown amounts of target DNA. A standard curve can be constructed by correlating initial template concentration in the standard samples with the number of PCR cycles (Ct) necessary to produce a specific threshold concentration of product. In the test samples, target PCR product accumulation can be measured after the same Ct, which allows interpolation of target DNA concentration from the standard curve. In some cases, quantitative PCR can be competitive quantitative PCR. In competitive quantitative PCR, an internal competitor DNA can be added at a known concentration to both serially diluted standard samples and unknown (environmental) samples. After co-amplification, ratios of the internal competitor and target PCR products can be calculated for both standard dilutions and unknown samples, and a standard curve can be constructed that plots competitor-target PCR product ratios against the initial target DNA concentration of the standard dilutions. Given equal amplification efficiency of competitor and target DNA, the concentration of the latter in environmental samples can be extrapolated from this standard curve. In some cases, quantitative PCR can be relative quantitative PCR. Relative quantitative PCR can determine the relative concentrations of specific nucleic acids. For example, reverse transcriptase PCR can be performed on mRNA species isolated from a subject. By determining that the concentration of a specific mRNA species varies, the method can determine whether the gene encoding the specific mRNA species is differentially expressed. Quantitative PCR can be used to measure level of DNA or RNA in a sample. In some cases, a profile of polynucleotides can be measured using a microarray. For example, a profile of polynucleotides can be measured by a genomic scan using a genomic microarray.

The nucleic acid analysis method can also include a sequencing step. A sequencing step can be used to identify and/or quantify the polynucleotides analyzed by other methods herein. Sequencing can be performed by basic sequencing methods, including Maxam-Gilbert sequencing, chain-termination sequencing, shotgun sequencing or Bridge PCR. Sequencing can also be performed by massively parallel sequencing methods, including high-throughput sequencing, pyro-sequencing, sequencing-by-synthesis, single-molecule sequencing, nanopore sequencing, semiconductor sequencing, sequencing-by-ligation, sequencing-by-hybridization, RNA-Seq (Illumina), Digital Gene Expression (Helicos), Next generation sequencing, Single Molecule Sequencing by Synthesis (SMSS)(Helicos), massively-parallel sequencing, Clonal Single Molecule Array (Solexa), shotgun sequencing, Maxam-Gilbert or Sanger sequencing, primer walking, sequencing using Illumina, PacBio, SOLiD, Ion Torrent, 454, or nanopore platforms.

The expression of a group of biomarkers in a sample can be measured by contacting a panel of probes with a sample, where the probes bind to one or more biomarkers of the group of biomarkers. In some cases, one probe can bind to multiple biomarkers in the group of biomarkers. In some cases, one probe can specifically bind to only one particular biomarker in the group of biomarkers. In some cases, the panel of probes can bind to all biomarkers in the group of biomarkers. In some cases, the panel of probes can bind some, but not all, of the biomarkers in the group of biomarkers. In some cases, the panel of probes can bind to molecules derived from the biomarkers. For example, the probes can bind to DNA derived (e.g., reversely transcribed) from the RNA (e.g., mRNA or miRNA) of the biomarkers.

The expression of a group of biomarkers can be measured using an assay. The assay can be any nucleic acid analysis method or polypeptide analysis method disclosed herein. In some cases, the assay can be a combination of any nucleic acid method and polypeptide analysis method disclosed herein. The assay can be PCR, an immunoassay, or a combination thereof. The assay can be any type of PCR used in nucleic acid analysis disclosed herein. For example, the PCR can be a quantitative reverse transcription polymerase chain reaction. The assay can be an immunoassay. Examples of immunoassays include immunoprecipitation, particle immunoassays, immunonephelometry, radioimmunoassays, enzyme immunoassays (e.g., ELISA), fluorescent immunoassays, chemiluminescent immunoassays, and Western blot analysis.

Expression patterns of biomarkers can be determined by statistical analysis. In some cases, an expression pattern of biomarkers can be measured by statistical regression. In another example, an expression pattern of biomarkers can be a multiple score of a first biomarker expression and a second biomarker expression. For example, the multiple score of biomarker 1×biomarker 2. In another example, an expression pattern of biomarkers can be a multiple score of a first biomarker expression and a second biomarker expression, where the first and second biomarkers are in the same or different treatment group and/or disease group. In another example, an expression pattern of biomarkers can be a ratio of a first biomarker expression to a second biomarker expression. In another example, an expression pattern of biomarkers can be a ratio of a first biomarker expression to a second biomarker expression, where the first and second biomarkers are in the same or different treatment group and/or disease group. In some aspects, the ratio of a first biomarker expression to a second biomarker expression can be in a range from about 0.01 to about 10000. In some aspects, the ratio of a first biomarker expression to a second biomarker expression can be at least about 0.5, 1, 1.5, 2, 3, 4, 5, 6, 7, 8, 9, 10, 20, 30, 40, 50, 60, 70, 80, 90, 100, 200, 500, or at least 1000. In another example, an expression pattern of biomarkers can be determined by multivariate statistical analysis. The multivariate statistical analysis may be principal component analysis, discriminant analysis, principal component analysis with discriminant analysis, partial least squares, partial least squares with discriminant analysis, canonical correlation, kernel principal component analysis, non-linear principal component analysis, factor analysis, multidimensional scaling, and cluster analysis. In another example, an expression pattern of biomarkers can be determined by principal components analysis. In another example, an expression pattern of biomarkers can be determined by machine learning and or pattern recognition.

The presence or level of a biomarker can be measured using any suitable immunoassay, for example, enzyme-linked immunoassays (ELISA), radioimmunoassays (RIAs), competitive binding assays, and the like. Specific immunological binding of an antibody to the biomarker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The analysis of a plurality of biomarkers can be carried out separately or simultaneously with one test sample. For separate or sequential assay of biomarkers, suitable apparatuses can include clinical laboratory analyzers such as the ELECSYS® (Roche), the AXSYM® (Abbott), the ACCESS® (Beckman), the ADVIA® CENTAUR® (Bayer) immunoassay systems, the NICHOLS ADVANTAGE® (Nichols Institute) immunoassay system, etc. Apparatuses or protein chips or gene chips can perform simultaneous assays of a plurality of biomarkers on a single surface. Useful physical formats comprise surfaces having a plurality of discrete, addressable locations for the detection of a plurality of different analytes. Such formats can include protein microarrays, or “protein chips” (see, e.g., Ng and Ilag, J. Cell Mol. Med. 6: 329-340 (2002)) and certain capillary devices (see e.g., U.S. Pat. No. 6,019,944). In these embodiments each discrete surface location can comprise antibodies to immobilize one or more analyte(s) (e.g., a biomarker) for detection at each location. Surfaces can alternatively comprise one or more discrete particles (e.g., microparticles or nanoparticles) immobilized at discrete locations of a surface, where the microparticles comprise antibodies to immobilize one analyte (e.g., a biomarker) for detection. The protein biochips can further include, for example, protein biochips produced by Ciphergen Biosystems, Inc. (Fremont, Calif.), Packard BioScience Company (Meriden Conn.), Zyomyx (Hayward, Calif.), Phylos (Lexington, Mass.) and Biacore (Uppsala, Sweden). Examples of such protein biochips are described in the following patents or published patent applications: U.S. Pat. No. 6,225,047; PCT International Publication No. WO 99/51773; U.S. Pat. No. 6,329,209, PCT International Publication No. WO 00/56934 and U.S. Pat. No. 5,242,828, each of which is incorporated by reference herein in its entirety.

Identifying biomarkers of ischemic stroke can comprise analyzing a profile of polynucleotides from a sample from a subject with a BBB disruption. Analyzing a profile of polynucleotides can comprise comparing the profile of polynucleotides to a polynucleotides reference profile. In some cases, comparing a profile of polynucleotides to a reference profile can comprise determining expression level differences between the polynucleotides in the ischemic stroke sample and the polynucleotides in the reference profile. When the expression level of a polynucleotide in sample from a subject with a BBB disruption is up-regulated or down-regulated compared to the expression level of the polynucleotide in a reference profile, the polynucleotide can be identified as a biomarker. The biomarker can be associated with BBB disruption. In some cases, further analysis can be carried out to identify the biomarker as a biomarker of BBB disruption. A polynucleotide can be identified as a biomarker when an expression level difference in the polynucleotide of at least 0.5, 1, 1.5, 2, 3, 4, 5, 6, 7, 8, 9, 10, 20, 30, 40, 50, 60, 70, 80, 90, or at least 100 fold can be detected in sample from a subject with a BBB disruption when compared to a polynucleotide reference profile. In some cases, a polynucleotide can be identified as a biomarker when an expression level difference in the polynucleotide increases at least 0.5, 1, 1.5, 2, 3, 4, 5, 6, 7, 8, 9, 10, 20, 30, 40, 50, 60, 70, 80, 90, or at least 100 fold in sample from a subject with a BBB disruption when compared to a polynucleotide reference profile. In some cases, a polynucleotide can be identified as a biomarker when an expression level difference in the polynucleotide decreases at least 0.5, 1, 1.5, 2, 3, 4, 5, 6, 7, 8, 9, 10, 20, 30, 40, 50, 60, 70, 80, 90, or at least 100 fold in sample from a subject with a BBB disruption when compared to a polynucleotide reference profile.

In some aspects, analyzing a profile of biomarkers may comprise using multivariate statistical analysis.

Biomarkers of BBB disruption can be identified using methods such as machine learning and or pattern recognition. In some cases, biomarkers of ischemic stroke or BBB disruption can be identified by based on a predictive model. Established statistical algorithms and methods useful as models or useful in designing predictive models, can include but are not limited to: analysis of variants (ANOVA); Bayesian networks; boosting and Ada-boosting; bootstrap aggregating (or bagging) algorithms; decision trees classification techniques, such as Classification and Regression Trees (CART), boosted CART, Random Forest (RF), Recursive Partitioning Trees (RPART), and others; Curds and Whey (CW); Curds and Whey-Lasso; dimension reduction methods, such as principal component analysis (PCA) and factor rotation or factor analysis; discriminant analysis, including Linear Discriminant Analysis (LDA), Eigengene Linear Discriminant Analysis (ELDA), and quadratic discriminant analysis; Discriminant Function Analysis (DFA); factor rotation or factor analysis; genetic algorithms; Hidden Markov Models; kernel based machine algorithms such as kernel density estimation, kernel partial least squares algorithms, kernel matching pursuit algorithms, kernel Fisher's discriminate analysis algorithms, and kernel principal components analysis algorithms; linear regression and generalized linear models, including or utilizing Forward Linear Stepwise Regression, Lasso (or LASSO) shrinkage and selection method, and Elastic Net regularization and selection method; glmnet (Lasso and Elastic Net-regularized generalized linear model); Logistic Regression (Log Reg); meta-learner algorithms; nearest neighbor methods for classification or regression, e.g. Kth-nearest neighbor (KNN); non-linear regression or classification algorithms; neural networks; partial least square; rules based classifiers; shrunken centroids (SC); sliced inverse regression; Standard for the Exchange of Product model data, Application Interpreted Constructs (StepAIC); super principal component (SPC) regression; and, Support Vector Machines (SVM) and Recursive Support Vector Machines (RSVM), among others. Additionally, clustering algorithms can also be used in determining subject sub-groups. In some cases, classification methods can be used to identify biomarkers of ischemic stroke or BBB disruption. Such classification methods include support vector machine (SVM), k-nearest neighbors (kNN), and classification trees (Hastie, et al. (2001) The Elements of Statistical Learning, Springer, N.Y.). 10-fold cross validation can be used to evaluate the classification accuracy.

In some cases, biomarkers of BBB disruption can be identified using Genetic Algorithm-K Nearest Neighbors (GA/kNN), a pattern recognition approach designed to identify sets of predictive variables which can optimally discriminate between classes of samples. Analysis of high dimensional genomic datasets using the GA/kNN method has been successfully used in fields such as cancer biology and toxicology to identify diagnostically relevant biomarker panels with powerful predictive ability.

The GA/kNN approach can combine a powerful search heuristic, GA, with a non-parametric classification method, kNN. In GA/kNN analysis, a small combination of genes (referred to as a chromosome) can be generated by random selection from the total pool of gene expression data. The ability of this randomly generated chromosome to predict sample class can be then evaluated using kNN. In this kNN evaluation, each sample can be plotted as a vector in an n^{th} dimensional space, with the coordinates of each vector being comprised of the expression levels of the genes of the chromosome. The class of each sample can be then predicted based on the majority class of the other samples which lie closest in Euclidian distance, which can be referred to the nearest neighbors. The predictive ability of the chromosome can be quantified as a fitness score, or the proportion of samples which the chromosome can be correctly able to predict. A termination cutoff (minimum proportion of correct predications) can determine the level of fitness required to pass evaluation. A chromosome which passes kNN evaluation can be identified as a near-optimal solution and can be recorded, while a chromosome which fails evaluation can undergo mutation and can be re-evaluated. This process of mutation and re-evaluation can be repeated until the fitness score of the chromosome exceeds the termination cutoff. This process can be repeated multiple times (typically thousands) to generate a pool of heterogeneous near-optimal solutions. The predicative ability of each gene in the total pool of gene expression can be then ranked according to the number of times it may be part of a near-optimal solution. The collective predictive ability of the top ranked genes can then be tested in a leave one out cross validation.

As used herein, the terms “reference” and “reference profile” can be used interchangeably to refer to a profile (e.g., expression) of biomolecules in a reference subject. A reference or a reference subject can be a control or a control subject respectively. In some cases, a reference can be the expression of a group of biomarkers in a reference subject. A reference or reference profile can be a profile of polynucleotides or a profile of polypeptides in a reference subject. In some cases, a reference subject can be a subject who has been previously diagnosed with a disruption of a BBB (such as through detection of HARM, intermediate HARM or severe HARM). In some cases, a reference subject can be a subject that does not have a disruption of a BBB. In some cases, a reference subject can be a subject who is a stroke subject. In some cases, the subject can be an ischemic stroke subject. In some cases, a reference subject can be a non-ischemic stroke subject. In some cases, a non-ischemic stroke can be a subject who has no ischemic stroke but has a transient ischemic attack, a non-ischemic stroke, or a stroke mimic. A subject having a non-ischemic stroke can have hemorrhagic stroke. When comparing profiles of polynucleotides and/or polypeptides in an BBB disruption subject to profiles of the biomolecules in a reference subject, the following groups of subjects can be used: (1) ischemic stroke; (2) hemorrhagic stroke; (3) normal; (4) TIAs; (5) other stroke mimics; (6) BBB disruption. One can measure profiles of biomolecules for all the subjects. Then, the members of any one of these groups can be compared to the profiles of the members of any other of these groups to define a function and weighting factor that best differentiates these groups based on the measured profiles. This can be repeated as all 5 groups are compared pairwise. A reference profile can be stored in computer readable form. In some aspects, a reference profile can be stored in a database or a server. In some cases, a reference can be stored in a database that can be accessible through a computer network (e.g., Internet). In some cases, a reference can be stored and accessible by Cloud storage technologies.

A biomarker disclosed herein can be identified as a biomarker of BBB disruption with further analysis. In some cases, a polynucleotide biomarker that is up-regulated in a sample from a subject with a BBB disruption compared to a reference profile can be identified as a biomarker of BBB disruption. In some cases, a polynucleotide biomarker that is down-regulated in a sample from a subject with a BBB disruption compared to a reference profile can be identified as a biomarker of BBB disruption

Methods herein can further comprise determining the effectiveness of a given biomarker or a given group of biomarkers at determining a condition such as BBB disruption. Parameters to be measured include those described in Fischer et al., Intensive Care Med. 29: 1043-51, 2003, which is incorporated herein in its entirety. These parameters include sensitivity and specificity, predictive values, likelihood ratios, diagnostic odds ratios, and receiver operating characteristic (ROC) curve areas. One or a group of effective biomarkers can exhibit one or more of the following results on these various parameters: at least 75% sensitivity, combined with at least 75% specificity; ROC curve area of at least 0.7, at least 0.8, at least 0.9, or at least 0.95; and/or a positive likelihood ratio (calculated as sensitivity/(1-specificity)) of at least 5, at least 10, or at least 20, and a negative likelihood ratio (calculated as (1-sensitivity)/specificity) of less than or equal to 0.3, less than or equal to 0.2, or less than or equal to 0.1. The ROC areas can be calculated and used in determining the effectiveness of a biomarker as described in US Patent Application Publication No. 2013/0189243, which is incorporated herein in its entirety.

Methods, devices and kits provided herein can assess a condition such as BBB disruption in a subject with high specificity and sensitivity. As used herein, the term “specificity” can refer to a measure of the proportion of negatives that are correctly identified as such (e.g., the percentage of healthy people who are correctly identified as not having the condition). As used herein, the term “sensitivity” can refer to a measure of the proportion of positives that are correctly identified as such (e.g., the percentage of sick people who are correctly identified as having the condition). Methods, devices and kits provided herein can assess a condition (e.g., BBB disruption) in a subject with a specificity of at least about 75%, 80%, 85%, 90%, 95%, 96%, 97%, 98%, 99%, or 100%. Methods, devices and kits provided herein can assess a condition (e.g., BBB disruption) in a subject with a sensitivity of at least about 75%, 80%, 85%, 90%, 95%, 96%, 97%, 98%, 99%, or 100%. Methods, devices and kits provided herein can assess a condition (e.g., BBB disruption) in a subject with a specificity of at least about 70% and a sensitivity of at least about 70%, a specificity of at least about 75% and a sensitivity of at least about 75%, a specificity of at least about 80% and a sensitivity of at least about 80%, a specificity of at least about 85% and a sensitivity of at least about 85%, a specificity of at least about 90% and a sensitivity of at least about 90%, a specificity of at least about 95% and a sensitivity of at least about 95%, a specificity of at least about 96% and a sensitivity of at least about 96%, a specificity of at least about 97% and a sensitivity of at least about 97%, a specificity of at least about 98% and a sensitivity of at least about 98%, a specificity of at least about 99% and a sensitivity of at least about 99%, or a specificity of about 100% a sensitivity of about 100%.

Methods of assessing a condition in a subject herein can achieve high specificity and sensitivity based on the expression of various numbers of biomarkers. In some cases, the methods of assessing a condition in a subject can achieve a specificity of at least about 70% and a sensitivity of at least about 70%, a specificity of at least about 75% and a sensitivity of at least about 75%, a specificity of at least about 80% and a sensitivity of at least about 80%, a specificity of at least about 85% and a sensitivity of at least about 85%, a specificity of at least about 90% and a sensitivity of at least about 90%, a specificity of at least about 95% and a sensitivity of at least about 95%, a specificity of at least about 96% and a sensitivity of at least about 96%, a specificity of at least about 97% and a sensitivity of at least about 97%, a specificity of at least about 98% and a sensitivity of at least about 98%, a specificity of at least about 99% and a sensitivity of at least about 99%, or a specificity of 100% a sensitivity of 100% based on the expression of no more than 1, 2, 3, 4, 5, 6, 7, 8, 9, or 10 biomarkers. In some cases, the methods, devices and kits of assessing a condition in a subject can achieve a specificity of at least about 92% and a sensitivity of at least about 92%, a specificity of at least about 95% and a sensitivity of at least about 95%, a specificity of at least about 96% and a sensitivity of at least about 96%, a specificity of at least about 97% and a sensitivity of at least about 97%, a specificity of at least about 98% and a sensitivity of at least about 98%, a specificity of at least about 99% and a sensitivity of at least about 99%, or a specificity of about 100% and a sensitivity of about 100% based on the expression of two biomarkers. In some cases, the methods of assessing a condition in a subject can comprise measuring the expression of two or more of LAIR2, IL-8, CXCL5, RBP7, CCDC149, LY96, DDIT4, HPSE, E2F3, ADAM15, ACN9, TMEM176B, BIRC2, EMR2, DUSP1, HSPA1B, RNASE2, IDI1, SCOC, FAM65A, CD14, F2RL1, PCMTD1, SMEK2, or SDPR, and the method can achieve a specificity of at least 90% and a sensitivity of at least 90%, a specificity of at least 92% and a sensitivity of at least 92%, a specificity of at least 95% and a sensitivity of at least 95%, a specificity of at least 96% and a sensitivity of at least 96%, a specificity of at least 97% and a sensitivity of at least 97%, a specificity of at least 98% and a sensitivity of at least 98%, a specificity of at least 99% and a sensitivity of at least 99%, or a specificity of 100% and a sensitivity of 100%. In some cases, the methods of assessing a condition in a subject can comprise measuring the expression of two or more (e.g., five) of RBP7, CCDC149, DDIT4, E2F3, and ADAM15, and the method can achieve a specificity of at least 98% and a sensitivity of at least 98%.

Assessing BBB disruption can comprise distinguishing a subject displaying severe HARM from a healthy subject, or a subject displaying mild HARM. Methods, devices, and kits herein can achieve high specificity and sensitivity in distinguishing a subject with severe HARM from a healthy subject, and distinguishing the subject with severe HARM from a subject with mild HARM. For example, methods, devices, and kits herein can achieve a specificity of at least 92% and a sensitivity of at least 92%, a specificity of at least 95% and a sensitivity of at least 95%, a specificity of at least 96% and a sensitivity of at least 96%, a specificity of at least 97% and a sensitivity of at least 97%, a specificity of at least 98% and a sensitivity of at least 98%, a specificity of at least 99% and a sensitivity of at least 99%, or a specificity of 100% and a sensitivity of 100% in distinguishing a subject with severe HARM from a healthy subject, and meanwhile can achieve a specificity of at least 92% and a sensitivity of at least 92%, a specificity of at least 95% and a sensitivity of at least 95%, a specificity of at least 96% and a sensitivity of at least 96%, a specificity of at least 97% and a sensitivity of at least 97%, a specificity of at least 98% and a sensitivity of at least 98%, a specificity of at least 99% and a sensitivity of at least 99%, or a specificity of 100% and a sensitivity of 100% in distinguishing the subject with severe HARM stroke from a subject with mild HARM.

In some cases, methods of assessing BBB disruption (e.g., distinguish severe HARM from a healthy condition or condition of mild HARM) that can comprise measuring a level of cell-free nucleic acid can also achieve the specificity and sensitivity disclosed herein. For example, such methods can achieve a sensitivity of at least 80%, and a specificity of at least 75%, a sensitivity of at least 85%, and a specificity of at least 80%, a sensitivity of at least 90%, and a specificity of at least 85%, a sensitivity of at least 95%, and a specificity of at least 80%, a sensitivity of 100%, and a specificity of at least 85%, a sensitivity of 100%, and a specificity of at least 90%, a sensitivity of 100%, and a specificity of at least 95%, a sensitivity of 100%, and a specificity of 100%. In some cases, the specificity can be at least 50%, 60%, 70%, 80%, 90%. In some cases, the sensitivity can be at least 50%, 60%, 70%, 80%, 90%.

The presence or level of a biomarker disclosed herein can be used to identify a hemorrhagic transformation.

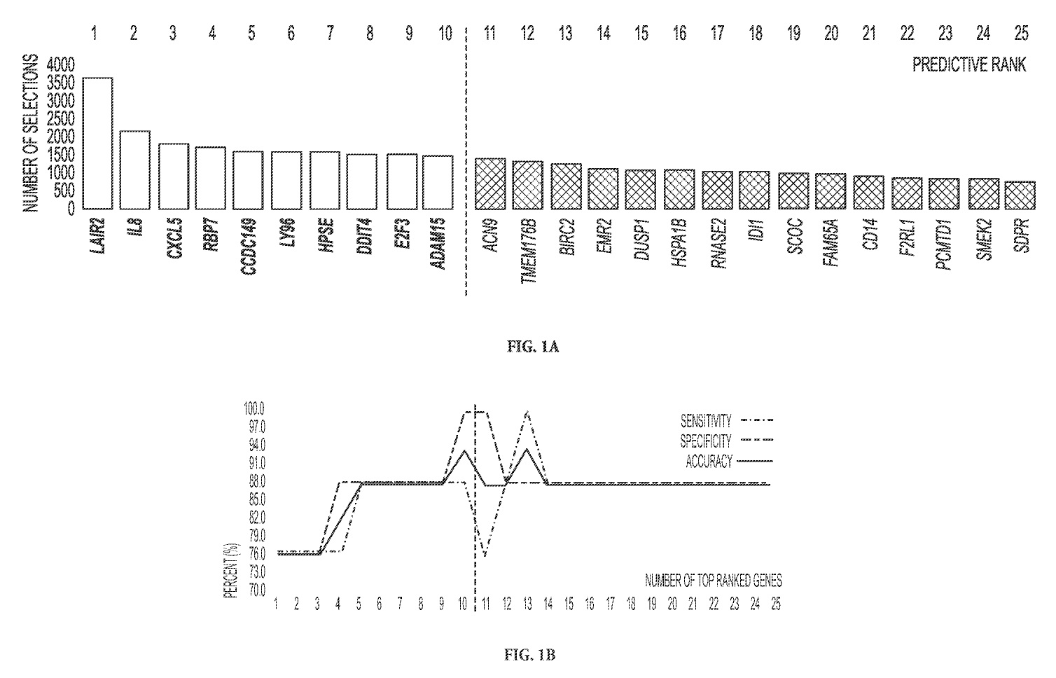

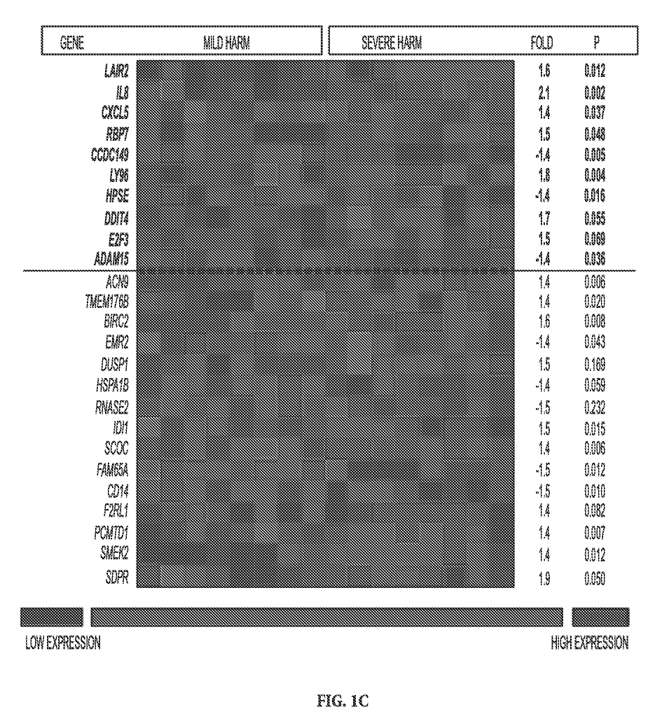

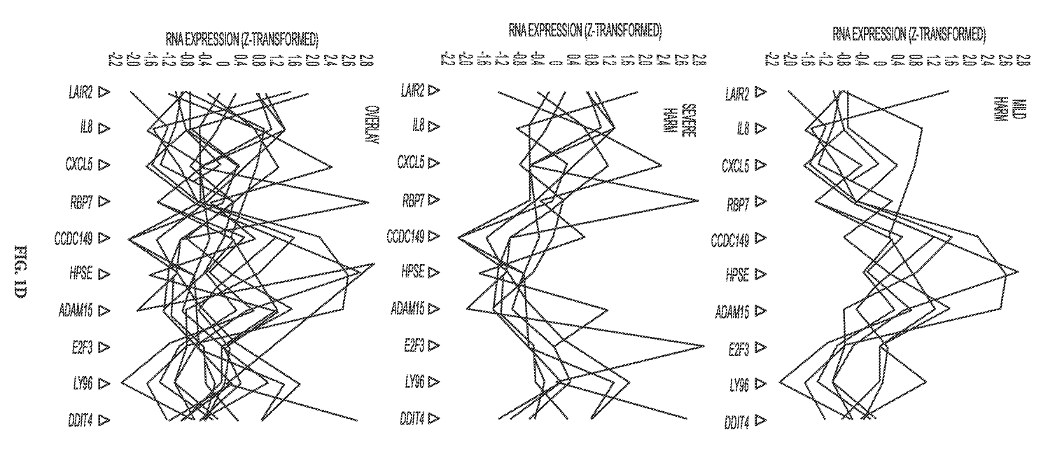

The presence of a biomarker for ischemic stroke can also be determined to assess a risk of developing ischemic stroke in addition to a BBB disruption. The biomarkers of ischemic stroke used to detect ischemic stroke can be any biomarkers of ischemic stroke identified by methods provided herein or known in the art. In some cases, the biomarkers of ischemic stroke (e.g., the first group of biomarkers of ischemic stroke) can include polynucleotides encoding at least one of CCL19, CCL21, Galectin 3, RAGE, ENA78, GMCSF, CD30, CCR7, CSPG2, IQGAP1, ORM1, ARG1, LY96, MMP9, CA4, s100A12, Nav3, SAA, IGα, IGγ, IGκ, IGλ, or an active fragment thereof. In some cases, the biomarkers of ischemic stroke (e.g., the second group of biomarkers of ischemic stroke) can include at least one of CCL19, CCL21, Galectin 3, RAGE, ENA78, GMCSF, CD30, CCR7, CSPG2, IQGAP1, ORM1, ARG1, LY96, MMP9, CA4, s100A12, Nav3, SAA, IGα, IGγ, IGκ, IGλ, or an active fragment thereof. In some cases, the biomarkers of ischemic stroke can include one or more cytokines. In some cases, the biomarkers of ischemic stroke (e.g., the first group of biomarkers of ischemic stroke) can include polynucleotides encoding at least one of BAFF, MMP9, APP, Aggrecan, Galectin 3, Fas, RAGE, Ephrin A2, CD30, TNR1, CD27, CD40, TNFα, IL6, IL8, IL10, IL1β, IFNγ, RANTES, IL1α, IL4, IL17, IL2, GMCSF, ENA78, IL5, IL12P70, TARC, GroAlpha, IL33, BLCBCA, IL31, MCP2, IGG3, IGG4, Isoform 2 of Teneurin 1, and isoform 2 of aDisintegrin or an active fragment thereof. In some cases, the biomarkers of ischemic stroke (e.g., the second group of biomarkers of ischemic stroke) can include at least one of BAFF, MMP9, APP, Aggrecan, Galectin 3, Fas, RAGE, Ephrin A2, CD30, TNR1, CD27, CD40, TNFα, IL6, IL8, IL10, IL1β, IFNγ, RANTES, IL1α, IL4, IL17, IL2, GMCSF, ENA78, IL5, IL12P70, TARC, GroAlpha, IL33, BLCBCA, IL31, MCP2, TLR2, TLR4, JAK2, CCR7, AKAP7, IL10, SYK, IL8, MyD88, CD3, CD4, IL22R, IL22, CEBPB or an active fragment thereof. In some cases, biomarkers of ischemic stroke provided herein can include at least one biomarkers in Table 1, FIG. 1A or any active form thereof. In some cases, biomarkers of ischemic stroke provided herein can include polynucleotides encoding at least one biomarkers in Table 1, FIG. 1A or any active form thereof.

The profiles of biomarkers of ischemic stroke can comprise a profile of at least one biomarkers of ischemic stroke disclosed herein. In some cases, the method can comprise measuring a profile of at least 1, 2, 3, 4, 5, 6, 7, 8, 9, 10, 20, 30, 40, 50, 60, 70, 80, 90, 100, 200, 300, 400, 500, 600, 700, 800, 900, or 1000 biomarkers of ischemic stroke, where the biomarkers of ischemic stroke are polynucleotides, and/or measuring a profile of at least 1, 2, 3, 4, 5, 6, 7, 8, 9, 10, 20, 30, 40, 50, 60, 70, 80, 90, 100, 200, 300, 400, 500, 600, 700, 800, 900, or 1000 biomarkers of ischemic stroke, where the biomarkers of ischemic stroke are polypeptides. In some cases, the method can comprise measuring the profiles of the same number of polynucleotide biomarkers of ischemic stroke and polypeptide biomarkers of ischemic stroke. In some cases, the method can comprise measuring the profiles of different numbers of polynucleotide biomarkers of ischemic stroke and polypeptide biomarkers of ischemic stroke. In some cases, the method of detecting ischemic stroke can comprise measuring a profile of polynucleotides encoding one or more of LY96, ARG1, and CA4, and/or measuring a profile of one or more of LY96, ARG1, and CA4. In some cases, the method of detecting ischemic stroke can comprise measuring a profile of polynucleotides encoding one or more of CCR7, CSPG2, IQGAP1, and ORM1, and/or measuring a profile of one or more of CCR7, CSPG2, IQGAP1, and ORM1. In some cases, the method of detecting ischemic stroke can comprise measuring a profile of polynucleotides encoding one or more of CCR7, CSPG2, IQGAP1, and ORM1, and/or measuring a profile of one or more of CCR7, CSPG2, IQGAP1, and ORM1. In some cases, the method of detecting ischemic stroke can comprise measuring a profile of polynucleotides encoding one or more of CCR7, CSPG2, IQGAP1, ARG1, LY96, MMP9, CA4, and s100A12 and ORM1, and/or measuring a profile of one or more of CCR7, CSPG2, IQGAP1, ARG1, LY96, MMP9, CA4, and s100A12 and ORM1.

Methods herein can further comprise administering a treatment for a condition. For instance, a method can comprise administering a treatment of a BBB disruption. In some embodiments, a method can comprise administering a treatment for a condition associated with a BBB disruption. For instance, a method can comprise administration of a treatment for, for example, meningitis, brain abscess, epilepsy, multiple sclerosis, neuromylelitis optica, neurological trypanosomiasis, progressive multifocal leukoencephalopathy, de vivo disease, Alzheimer's disease, cerebral edema, a prior disease, encephalitis, and/or rabies. Treatments can include anticonvulsants, antihypertensive agents, osmotic diuretics or a combination thereof. Examples of treatments can further include an antibiotic such as daptomycin, dalbavancin, ceftobiprole, ceftaroline, clindamycin, linezolid, mupirocin, oritavancin, tedizolid, telavancin, tigecycline, a carbapenem, ceftazidime, cefepime, ceftobiprole, a fluoroquinolone, piperacillin, ticarcillin, linezolid, a streptogramin, tigecycline, daptomycin, cephalosporin, vancomycin, amphotericin B; an anti-epileptic drug; lipoic acid; an immunosuppressant; a narcotic such as fentanyl, morphine, methadone, etorphine, levophanol, sufentanil, D-Ala², N-MePhe⁴, Gly-ol]-enkephalin (DAMGO), butophanol, buprenorphine, naloxone, naltrexone, D-Phe-Cys-Tyr-D-Trp-Orn-Thr-Pen-Thr-NH (CTOP), iprenorphine, b-funaltrexamine, naloxonazine, nalorphine, pentazocine, nalbuphine, codeine, hydrocodone, oxycodone, nalmephene; an anti-inflammatory such as diclofenac, ketoprofen, ibuprofen, aspirin; a salt of any of these; and combinations of any of these.

A method can comprise administering a treatment of an ischemic stroke to a subject deemed at risk of developing ischemic stroke. In some cases, the methods can comprise administering a pharmaceutically effective dose of a drug or a salt thereof for treating ischemic stroke. In some embodiments, a drug for treating ischemic stroke can comprise a thrombolytic agent or antithrombotic agent. In some embodiments, a drug for treating ischemic stroke can be one or more compounds that are capable of dissolving blood clots such as psilocybin, tPA (Alteplase or Activase), reteplase (Retavase), tenecteplase (TNKasa), anistreplase (Eminase), streptoquinase (Kabikinase, Streptase) or uroquinase (Abokinase), and anticoagulant compounds, i.e., compounds that prevent coagulation and include, without limitation, vitamin K antagonists (warfarin, acenocumarol, fenprocoumon and fenidione), heparin and heparin derivatives such as low molecular weight heparins, factor Xa inhibitors such as synthetic pentasaccharides, direct thrombin inhibitors (argatroban, lepirudin, bivalirudin and ximelagatran) and antiplatelet compounds that act by inhibition of platelet aggregation and, therefore, thrombus formation and include, without limitation, cyclooxygenase inhibitors (aspirin), adenosine diphosphate receptor inhibitors (clopidrogrel and ticlopidine), phosphodiesterase inhibitors (cilostazol), glycoprotein inhibitors (Abciximab Eptifibatide, Tirofiban and Defibrotide) and adenosine uptake inhibitors (dipitidamol). The drug for treating ischemic stroke can be tissue plasminogen activator (tPA).

In some cases, a treatment can comprise endovascular therapy. In some cases, endovascular therapy can be performed after a treatment is administered. In some cases, endovascular therapy can be performed before a treatment is administered. In some cases, a treatment can comprise a thrombolytic agent In some cases, an endovascular therapy can be a mechanical thrombectomy. In some cases, a stent retriever can be sent to the site of a blocked blood vessel in the brain to remove a clot. In some cases, after a stent retriever grasps a clot or a portion thereof, the stent retriever and the clot or portions thereof can be removed. In some cases, a catheter can be threaded through an artery up to a blocked artery in the brain. In some cases, a stent can open and grasp a clot or portions thereof, allowing for the removal of the stent with the trapped clot or portions thereof. In some cases, suction tubes can be used. In some cases, a stent can be self-expanding, balloon-expandable, and or drug eluting.

In some cases, the treatments disclosed herein may be administered by any route, including, without limitation, oral, intravenous, intramuscular, intra-arterial, intramedullary, intrathecal, intraventricular, transdermal, subcutaneous, intraperitoneal, intranasal, enteric, topical, sublingual or rectal route. A review of the different dosage forms of active ingredients and excipients to be used and their manufacturing processes is provided in “Tratado de Farmacia Galénica”, C. Fault and Trillo, Luzán 5, S. A. de Ediciones, 1993 and in Remington's Pharmaceutical Sciences (A. R. Gennaro, Ed.), 20^{th} edition, Williams & Wilkins PA, USA (2000). Examples of pharmaceutically acceptable vehicles are known in prior art and include phosphate buffered saline solutions, water, emulsions, such as oil/water emulsions, different types of humectants, sterile solutions, etc. The compositions that comprise said vehicles may be formulated by conventional processes which are known in prior art.

In some cases, the methods can comprise administering a pharmaceutically effective dose of a drug for treating ischemic stroke within 24 hours, 12 hours, 11 hours, 10 hours, 9 hours, 8 hours, 7 hours, 6 hours, 5 hours, 4 hours, 3 hours, 2 hours, or 1 hour, 30 minutes, 20 minutes, or 10 minutes from the ischemic stroke onset. For example, the methods can comprise administering a pharmaceutically effective dose of a drug for treating ischemic stroke within 4.5 hours of ischemic stroke onset. In a particular example, the methods can comprise administering a pharmaceutically effective dose of tPA within 4.5 hours of ischemic stroke onset. In some cases, the methods can comprise determining whether or not to take the patient to neuro-interventional radiology for clot removal or intra-arterial tPA. In this particular example, the methods can comprise administering a pharmaceutically effective dose of intra-arterial tPA within 8 hours of ischemic stroke onset. In certain cases, the methods comprise administering a treatment to the subject if the level of the cell-free nucleic acids in the subject can be higher than a reference level. In some embodiments, a treatment may not be administered if the level of the cell-free nucleic acids in the subject is equal to or less than the reference. In some embodiments, a treatment can be administered if ischemic stroke, or BBB disruption is determined. In some cases, an identification of hemorrhagic transformation or BBB disruption can prevent the administration of a treatment, for example tPA.

A drug for treating BBB disruption or ischemic stroke can alter the expression of one or more biomarkers in a subject receiving the drug. In some cases, the drug for treating a disease or condition described herein can at least partially increase the expression, function, or both of one or more biomarkers in a subject receiving the drug. In some cases, the drug for treating a disease or condition described herein can at least partially reduce or suppress the expression, function, or both of one or more biomarkers in a subject receiving the drug.

### Kits

Provided herein are kits for detecting a disease or condition, for example, BBB disruption in a subject. A kit can be used for performing any methods described herein. For example, the kits can be used to determine a presence or level of a biomarker described herein in a subject. A kit can be used to assess a disruption of a BBB, or a condition associated therewith. When assessing the condition with a kit, high specificity and sensitivity can be achieved. The kits can also be used to evaluate a treatment of a condition associated with BBB disruption. For example, kits disclosed herein can comprise a panel of probes and a detecting reagent.

The kits can comprise a probe for measuring a panel of one or more biomarkers in a sample from a subject. The probe can bind (e.g., directly or indirectly) to at least one biomarker in the sample. For example, a probe can hybridize to a nucleic acid biomarker that can be present in the sample. In some cases, the kits can comprise a probe for measuring a level of nucleic acids such as cell-free nucleic acids in a sample from the subject, where the probe binds or hybridizes to the nucleic acids. The kit can further comprise a detecting reagent to examining the binding of the probe to at least one of the nucleic acids. In some cases, a probe can determine a presence of any one or all of LAIR2, RBP7, CCDC149, DDIT4, E2F3, ADAM15, IL-8, CXCL5, LY96, HPSE, ACN9, TMEM176B, BIRC2, EMR2, DUSP1, HSPA1B, RNASE2, IDI1, SCOC, FAM65A, CD14, F2RL1, PCMTD1, SMEK2, and SDPR.

The kits can comprise a plurality of probes that can detect one or more biomarkers of BBB disruption. In some cases, the kits can comprise a panel of probes for detecting a group of biomarkers of BBB disruption. In some cases, the kits can comprise a panel of probes for detecting a first group of biomarkers of BBB disruption and a second group of biomarkers for a condition associated with a disruption of a BBB (e.g. ischemic stroke). In some cases, the first group of biomarkers can comprise a first class of biomolecules and the second group of biomarkers can comprise a second class of biomolecules. In some cases, the first and second class of biomolecules can be different classes of biomolecules. For example, the first class of biomolecules can be polynucleotides. In another example, the second class of biomolecules can be polypeptides. In another example, the first class of biomolecules can be polynucleotides and the second class of biomolecules can be polypeptides.

The kits can comprise one or more probes that can bind one or more biomarkers of BBB disruption. In some cases, the probes can be oligonucleotides capable of binding to the biomarkers of BBB disruption. The biomarkers of BBB disruption bounded by the oligonucleotides can be polynucleotides, polypeptides or proteins. In some cases, the probes in the kits can be oligonucleotides capable of hybridizing to at least one of the biomarkers of BBB disruption. The oligonucleotides can be any type of nucleic acids including DNA, RNA or hybridization thereof. The oligonucleotides can be any length. In some cases, the probes herein can be other types of molecules, including aptamers.

The probes can also be proteinaceous materials, e.g., polypeptides or polypeptide fragments of exemplary biomarkers. In some cases, the probe may be a proteinaceous compound. There is a wide variety of protein-protein interactions; however, proteins also bind nucleic acids, metals and other non-proteinaceous compounds (e.g., lipids, hormones, transmitters). Some other examples of proteins that may be used as either targets or probes include antibodies, enzymes, receptors, and DNA- or RNA-binding proteins. Both antibody and antigen preparations can be in a suitable titrated form, with antigen concentrations and/or antibody titers given for easy reference in quantitative applications.

The probes can be antibodies capable of specifically binding at least one of the biomarkers of BBB disruption. An antibody that “specifically binds to” or is “specific for” a particular polypeptide or an epitope on a particular polypeptide can be one that binds to that particular polypeptide or epitope on a particular polypeptide without substantially binding to any other polypeptide or polypeptide epitope. Alternatively, an antibody that specifically binds to an antigen can refer to the binding of an antigen by an antibody or fragment thereof with a dissociation constant (IQ) of 10⁴ or lower, as measured by a suitable detection instrument, e.g., surface plasmon resonance analysis using, for example, a BIACORE® surface plasmon resonance system and BIACORE® kinetic evaluation software (e.g. version 2.1). The affinity or dissociation constant (K_{d}) for a specific binding interaction can be preferably about 500 nM or lower, more preferably about 300 nM or lower and preferably at least 300 nM to 50 pM, 200 nM to 50 pM, and more preferably at least 100 nM to 50 pM, 75 nM to 50 pM, 10 nM to 50 pM.

The probes can be labeled. For example, the probes can comprise labels. The labels can be used to track the binding of the probes with biomarkers of blood brain barrier disruption in a sample. The labels can be fluorescent or luminescent tags, metals, dyes, radioactive isotopes, and the like. Examples of labels include paramagnetic ions, radioactive isotopes; fluorochromes, metals, dyes, NMR-detectable substances, and X-ray imaging compounds. Paramagnetic ions include chromium (III), manganese (II), iron (III), iron (II), cobalt (II), nickel (II), copper (II), neodymium (II), samarium (III), ytterbium (III), gadolinium (III), vanadium (II), terbium (III), dysprosium (III), holmium (III) and/or erbium (III). Ions useful in other contexts, such as X-ray imaging, include but are not limited to lanthanum (III), gold (III), lead (II), and especially bismuth (III). Radioactive isotopes include ¹⁴-carbon, ¹⁵chromium, ³⁶-chlorine, ⁵⁷cobalt, and the like may be utilized. Among the fluorescent labels contemplated for use include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red. Enzymes (an enzyme tag) that will generate a colored product upon contact with a chromogenic substrate may also be used. Examples of suitable enzymes include urease, alkaline phosphatase, (horseradish) hydrogen peroxidase or glucose oxidase. Secondary binding ligands can be biotin and/or avidin and streptavidin compounds. The use of such labels is well known to those of skill in the art and is described, for example, in U.S. Pat. Nos. 3,817,837; 3,850,752; 3,939,350; 3,996,345; 4,277,437; 4,275,149 and 4,366,241; each incorporated herein by reference.

The probes disclosed herein can be used to measure the expression of a group of biomarkers in methods of assessing BBB disruption, a condition associated therewith, or a condition or disease described herein. In some cases, probes can be labeled probes that comprise any labels described herein. In some cases, the probes can be synthetic, e.g., synthesized in vitro. In some cases, the probes can be different from any naturally occurring molecules.

The probes can comprise one or more polynucleotides. In some cases, the probes can comprise polynucleotides that bind (e.g., hybridize) with the group of biomarkers. In some case, the probes can comprise polynucleotides that bind (e.g., hybridize) with the RNA (e.g., mRNA or miRNA) of the group of biomarkers. In some cases, the probes can comprise polynucleotides that bind (e.g., hybridize) with DNA derived (e.g., reversely transcribed) from RNA (e.g., mRNA or miRNA) of the group of biomarkers.

The probes can comprise polypeptides. In some cases, the probes can comprise polypeptides that bind to the proteins (or fragments of the proteins) of the group of biomarkers. Such probes can be antibodies or fragments thereof.

The probes can also comprise any other molecules that bind to the group of biomarkers other than polynucleotides or polypeptides. For example, the probes can be aptamers or chemical compounds. In some cases, the probes can comprise a combination of polynucleotides, polypeptides, aptamers, chemical compounds, and any other type of molecules.

The kits can further comprise a detecting reagent. The detecting reagent can be used for examining binding of the probes with the group of biomarkers. The detecting reagent can comprise any label described herein, e.g., a fluorescent or radioactive label. In some cases, the kits can also include an immunodetection reagent or label for the detection of specific immunoreaction between the provided biomarkers and/or antibody, as the case may be, and the diagnostic sample. Suitable detection reagents are well known in the art as exemplified by radioactive, enzymatic or otherwise chromogenic ligands, which are typically employed in association with the antigen and/or antibody, or in association with a second antibody having specificity for first antibody. Thus, the reaction can be detected or quantified by means of detecting or quantifying the label. Immunodetection reagents and processes suitable for application in connection with the novel methods disclosed herein are generally well known in the art.

The reagents can include ancillary agents such as buffering agents and protein stabilizing agents, e.g., polysaccharides and the like. The kit may further include where necessary agents for reducing background interference in a test, agents for increasing signal, apparatus for conducting a test, calibration curves and charts, standardization curves and charts, and the like.

The kits can further comprise a computer-readable medium for assessing a condition in a subject. For example, the computer-readable medium can analyze the difference between the expression of the group of biomarkers in a sample from a subject and a reference, thus assessing a condition in the subject. In some embodiments, a kit disclosed herein can comprise instructions for use.

### Systems for Detecting BBB Disruption

Disclosed herein are systems for assessing BBB disruption in a subject. Such systems can comprise a memory that stores executable instructions. The systems can further comprise a processor that executes the executable instructions to perform the methods disclosed herein.

Disclosed herein are systems for detecting BBB disruption, or a condition associated therewith, in a subject. The systems can comprise a memory that stores executive instruction and a processor that executes the executable instructions. The systems can be configured to perform any method of detecting BBB disruption disclosed herein.

In some embodiments, a system can be configured to communicate with a database. In some embodiments, a system can transmit data to a database or server. A database or server can be a cloud server or database. In some embodiments, a system can transmit data wirelessly via a Wi-Fi, or Bluetooth connection. Databases can include functional or bioinformatics databases such as the Database for Annotation, Visualization and Integrated Discovery (DAVID); BioGraph, Entrez, GeneCards, Genome Aggregation Database, mGEN, MOPED, SOURCE, Rfam, DASHR, UnitProt, Pfam, Swiss-Prot Protein Knowledgebase, Protein Data Bank (PDB), and Structural Classification of Proteins (SCOP).

In some aspects, a system described herein can comprise centralized data processing, that could be cloud-based, internet-based, locally accessible network (LAN)-based, or a dedicated reading center using pre-existent or new platforms.

FIG. 5 provides an exemplary illustration of a computer implement workflow.

Biomarkers in a sample from a subject can be detected using a probe in an assay as described herein. The assay output can be fed into a system that can compile a biomarker profile. In some cases, the system can compare the profile to a reference as described herein. A result can be stored via local or cloud based storage for future use, and/or can be communicated to the subject and/or a healthcare provider.

In some aspects, a system can comprise software. A software can rely on structured computation, for example providing registration, segmentation and other functions, with the centrally-processed output made ready for downstream analysis.

In some aspects, the software would rely on unstructured computation, artificial intelligence or deep learning. In a variation of this aspect, the software would rely on unstructured computation, such that data could be iteratively. In a further variation of this aspect, the software would rely on unstructured computation, so-called “artificial intelligence” or “deep learning.” For example, a method described herein such as GA/kNN can employ deep learning to generate near-optimal solutions of grouped data, which can be performed iteratively to improve predictive value of biomarkers.

The devices can comprise immunoassay devices for measuring profiles of polypeptides or proteins. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. These devices and methods can utilize labeled probes in various sandwiches, competitive or non-competitive assay formats, to generate a signal that can be related to the presence or amount of an analyte of interest. Additionally, certain methods and devices, such as biosensors and optical immunoassays, can be employed to determine the presence or amount of analytes without the need for a labeled molecule. See, e.g., U.S. Pat. Nos. 5,631,171; and 5,955,377, each of which is hereby incorporated by reference in its entirety, including all tables, figures and claims. One skilled in the art can also recognize that robotic instrumentation including but not limited to Beckman ACCESS®, Abbott AXSYM®, Roche ELECSYS®, Dade Behring STRATUS® systems are among the immunoassay analyzers that are capable of performing the immunoassays taught herein.

The devices can comprise a filament-based diagnostic device. The filament-based diagnostic device can comprise a filament support which provides the opportunity to rapidly and efficiently move probes between different zones (e.g., chambers, such as the washing chamber or a reporting chamber) of an apparatus and still retain information about their location. It can also permit the use of very small volumes of various samples—as little as nanoliter volume reactions. The filament can be constructed so that the probes are arranged in an annular fashion, forming a probe band around the circumference of the filament. This can also permit bands to be deposited so as to achieve high linear density of probes on the filament.

The filament can be made of any of a number of different materials. Suitable materials include polystyrene, glass (e.g., fiber optic cores), nylon or other substrate derivatized with chemical moieties to impart desired surface structure (3-dimensional) and chemical activity. The filament can also be constructed to contain surface features such as pores, abrasions, invaginations, protrusions, or any other physical or chemical structures that increase effective surface area. These surface features can, in one aspect, provide for enhanced mixing of solutions as the filament passes through a solution-containing chamber, or increase the number and availability of probe molecules. The filament can also contain a probe identifier which allows the user to track large numbers of different probes on a single filament. The probe identifiers may be dyes, magnetic, radioactive, fluorescent, or chemiluminescent molecules. Alternatively, they may comprise various digital or analog tags.

The probes that are attached to the filaments can be any of a variety of biomolecules, including, nucleic acid molecules (e.g., oligonucleotides) and antibodies or antibodies fragments. The probes should be capable of binding to or interacting with a target substance of interest (e.g., exemplary polypeptide biomarkers or their encoding mRNA molecules) in a sample to be tested (e.g., peripheral blood), such that the binding to or interaction can be capable of being detected.

### EXAMPLES

### Exemplary Study 1

Experimental Design:

Microarray was used to generate peripheral blood expression levels for over 10,000 genes and GA/kNN was used to identify a pattern of gene expression which could optimally discriminate between groups. Functional enrichment analysis via the Database for Annotation, Visualization and Integrated Discovery (DAVID) was then used to determine whether genes identified by GA/kNN were enriched for specific biological processes or signaling pathways.

### Example 1—Patient Selection

Acute ischemic stroke patients were recruited at Suburban Hospital, Bethesda, Md. AIS diagnosis was confirmed via MRI and all samples were collected within 24 hours of symptom onset, and prior to the administration of rTPA, Injury severity was determined according to the NIH stroke scale (NUBS) at the time of blood collection. Demographic information was collected from either subjects or significant others by a trained clinician. All procedures were approved by the institutional review boards of the National Institute of Neurological Disorders/National Institute on Aging at NIH and Suburban Hospital. Written informed consent was obtained from all subjects or their authorized representatives prior to any study procedures.

Peripheral blood samples were obtained from 34 acute ischemic patients, and blood brain barrier disruption was assessed via HARM on contrast MRI at two day follow up. Nine patients were identified as presenting with intermediate levels of HARM and were excluded. Four patients were excluded due to post-stroke hemorrhagic events. Of the 21 remaining patients, 8 patients exhibiting mild HARM and 8 patients exhibiting severe HARM were selected for analysis based on matching clinical and demographic characteristics.

Demographic and Clinical Characteristics:

Patients in the mild HARM and severe HARM groups were well matched in terms of cardiovascular disease risk factors, comorbidities, and medication status. Stroke severity via NIHSS was greater at ED admission in patients in the severe HARM group than in the mild HARM group, however, almost all patients presented with relatively low NIHSS scores. Table 2 below depicts exemplary patient profiles. A greater number of patients in the severe HARM group were administered rTPA than in the mild HARM group.

TABLE 2

| | | SEVERE | | |
|---|---|---|---|---|
| | | HARM | | |
| | MILD HARM | (n = 8) | STAT (df) | P |
| Age (mean ± SD) | 72.9 ± 11.8 | 75.4 ± 14.7 | t = 0.37 (14) | 0.714 |
| NIHSS (mean ± SD) | 1.0 ± 2.1 | 6.9 ± 5.8 | t = 2.70 (14) | 0.009* |
| Male n (%) | 3 (37.5) | 3 (37.5) | χ² = 0.00 (1) | 1.000 |
| Female n (%) | 5 (62.5) | 5 (62.5) | χ² = 0.00 (1) | 1.000 |
| Family history of stroke n (%) | 4 (50.0) | 5 (62.5) | χ² = 0.25 (1) | 0.614 |
| Hypertension n (%) | 5 (62.5) | 6 (75.0) | χ² = 0.29 (1) | 0.590 |
| Diabetes n (%) | 1 (12.5) | 2 (25.0) | χ² = 0.41 (1) | 0.522 |
| Atrial fibrillation n (%) | 1 (12.5) | 1 (12.5) | χ² = 0.00 (1) | 1.000 |
| Myocardial infarction n (%) | 2 (25.0) | 1 (12.5) | χ² = 0.41 (1) | 0.522 |
| Dyslipidemia n (%) | 5 (62.5) | 6 (75.0) | χ² = 0.29 (1) | 0.590 |
| Previous stroke n (%) | 0 (0.00) | 1 (12.5) | χ² = 1.07 (1) | 0.302 |
| Hypertension medication n (%) | 5 (62.5) | 6 (75.0) | χ² = 0.29 (1) | 0.590 |
| Diabetes medication n (%) | 1 (12.5) | 0 (0.00) | χ² = 1.07 (1) | 0.302 |
| Cholesterol medication n (%) | 5 (62.5) | 4 (50.0) | χ² = 0.25 (1) | 0.614 |
| Anticoagulant or antiplatelet n (%) | 5 (62.5) | 4 (50.0) | χ² = 0.25 (1) | 0.614 |
| rtPA n (%) | 0 (0.00) | 4 (50.0) | χ² = 5.33 (1) | 0.021* |
| Current smoker n (%) | 0 (0.00) | 0 (0.00) | χ² = 0.00 (1) | 1.000 |
| Previous smoker n (%) | 2 (25.0) | 5 (62.5) | χ² = 2.29 (1) | 0.131 |

*SIGNIFICANT

### Example 2: Magnetic Resonance Imaging (MRI)

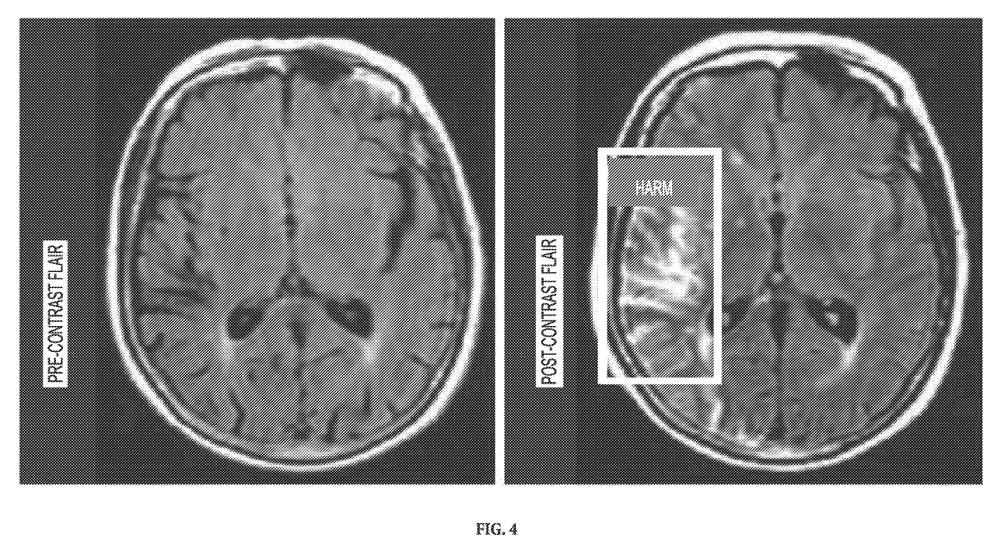

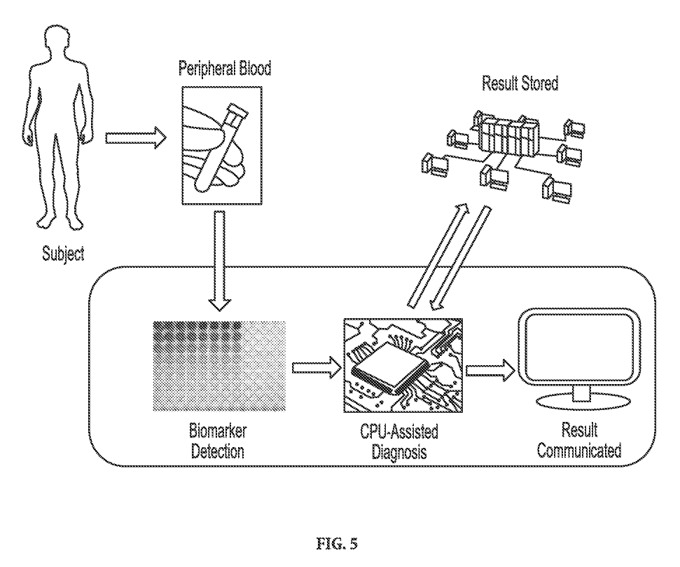

MRI was performed using a 1.5-Tesla clinical MR system during acute triage and at 2 d follow-up. The standardized protocol included: diffusion weighted imaging, T2*-weighted gradient-recalled echo (GRE), FLAIR, and perfusion-weighted imaging. Perfusion weighted imaging was obtained using a bolus passage of Gd-DTPA (0.1 mmol/kg). All FLAIR images were reviewed sequentially by expert readers in a randomized order, blinded to clinical information. GRE on day two was assessed for presence of hemorrhage. Post-contrast FLAIR on day two was assessed for location and level of HARM. HARM was identified positive when CSF intensity in the sulci or ventricles appeared hyperintense in comparison with initial examination (FIG. 4). Mild HARM was defined as hyperintense regions present on 0-5 MRI slices. Severe HARM was defined as linear and continuous hyperintense regions present in >10 MRI slices.

### Example 3: Blood Collection and RNA Extraction

Peripheral whole blood samples were collected via PAXgene RNA tubes (Qiagen, Valencia, Calif.) and stored at −80° C. until RNA extraction. Total RNA was extracted via the PreAnalytiX PAXgene blood RNA kit (Qiagen). Quantity and purity of isolated RNA was determined via spectrophotometry (NanoDrop, Thermo Scientific, Waltham, Mass.) and quality of RNA was confirmed by gel electrophoresis.

### Example 4: RNA Amplification and Microarray

RNA was amplified and biotinylated using the TotalPrep RNA amplification kit (Applied Biosystems, Grand Island, N.Y.). The TotalPrep RNA amplification kit was employed to generate biotinylated, amplified RNA for hybridization to the arrays. The procedure consisted of reverse transcription with an oligo (dT) primer and a reverse transcriptase designed to produce higher yields of first strand cDNA. The cDNA underwent a second strand synthesis and clean up to become a template for in vitro transcription. The in vitro transcription resulted in biotin labeled antisense cRNA copies of each mRNA in a sample.

Samples were hybridized to HumanRef-8 expression bead chips (Illumina, San Diego, Calif.) containing probes for transcripts originating from over 10,000 genes and scanned using the Illumina BeadStation. The expression beadchips are constructed by introducing oligonucleotide bearing 3-micron beads into microwells etched into the surface of a slide-sized silicon substrate. The beads self-assemble onto the beadchips resulting in an average of 30-fold redundancy of every full-length oligonucleotide. After random bead assembly, 29-mer address sequences present on each bead can be used to map the array, identifying the location of each bead.

Raw probe intensities were background subtracted, quantile normalized, and then summarized at the gene level using Illumina GenomeStudio. Sample labeling, hybridization, and scanning were performed per standard Illumina protocols.

### Example 5: GA/kNN Analysis

Genetic Algorithm-K Nearest Neighbors (GA/kNN) is a pattern recognition approach designed to identify sets of predictive variables which can optimally discriminate between classes of samples. Analysis of high-dimensional genomic datasets using the GA/kNN method has been successfully used in fields such as cancer biology and toxicology to identify diagnostically relevant biomarker panels with powerful predictive ability. Here the GA/kNN approach was applied to analyze peripheral blood gene expression data generated via microarray to identify transcriptional patterns which could potentially be used for the clinical identification of patients who can be at high risk of post-stroke blood brain barrier disruption.

GA/kNN combines a non-parametric classification method, kNN, with a powerful search heuristic, GA. KNN can be used to classify an unknown sample based on its Euclidian distance relative to training samples of known class when the training samples and the unknown sample are plotted in an nth dimensional space as vectors formed by the expression levels of n number of genes. The Euclidian distance between the unknown sample vector and each training sample vector can be calculated and a set of training samples which lie the shortest distance away from the unknown sample are identified as the nearest neighbors. The classes of the nearest neighbors can be used to predict the class of the unknown sample. Typically, the number of nearest neighbors (k) used for this type of application can range from 3-5 and the majority class of the nearest neighbors can be used to call the class of the unknown sample. FIG. 3B illustrates the application of kNN to predict the identity of an unknown sample using 2-dimensional vectors formed by the expression levels of two genes. In this example, the unknown sample can be classified as severe based on the class of its 5 nearest training samples.

The other component of GA/kNN, GA, is a stochastic optimization method based on principles of natural selection. As applied in this context, a combination of genes (chromosome or chromosome of data) can be randomly generated from the total pool of gene expression data and can be evaluated based on its ability to predict sample class using KNN where each sample can be treated and as an unknown once (the remaining samples constitute the training samples) in a leave one out paradigm. The predictive ability of the chromosome can be quantified as a fitness score, or the proportion of samples which the chromosome can be correctly able to predict. A termination cutoff can be set (minimum proportion of correct predications) which determines the level of fitness required to pass evaluation. A chromosome which passes kNN evaluation can be added to the pool of near optimal solutions, while a chromosome which fails evaluation undergoes mutation and can be re-evaluated. This process of mutation and re-evaluation can be repeated until the fitness score of the chromosome exceeds the termination cutoff and can be added to the pool of near optimal solutions (FIG. 3A). This process can be repeated multiple times (typically thousands) to generate a pool of unique near-optimal solutions. The predicative ability of each gene in the total pool of gene expression can then be ranked according to the number of times it was part of an optimal solution. The predictive ability of the top ranked variables can then be tested in a leave one out cross validation.

Normalized microarray data were filtered based on absolute fold difference between stroke and control regardless of statistical significance; genes exhibiting a greater than 1.4 absolute fold difference in expression between MS and control were retained for analysis. Filtered gene expression data were z-transformed and GA/kNN analysis was performed using source code developed by Li et al. Two-thousand near-optimal solutions were collected per sample using five nearest neighbors, majority rule, a chromosome length of 5, and a termination cutoff of 0.85. Leave one out cross validation was performed using the top 25 ranked gene products.

The top 25 transcripts most predictive of the development of severe HARM ranked by GA/kNN, as ordered by the number of times each transcript was selected as part of a near optimal solution, are listed in FIG. 1A. The accession numbers of these genes are listed below in Table 3.

TABLE 3

| | Accession No. | Accession No. |
|---|---|---|
| Gene name | (mRNA) | (protein) |
| Leukocyte associated | NM_002288.5 | NP 002279.2 |
| immunoglobulin like | | |
| receptor 2 (LAIR2) | | |
| Interleukin 8 (IL8) | NM_000584.3 | NP 000575.1 |
| C—X—C motif chemokine | NM_002994.4 | NP 002985.1 |
| ligand 5 (CXCL5) | | |
| Retinol binding protein 7 | NM_052960.2 | NP 443192.1 |
| (RBP7) | | |
| Coiled-coil domain | NM_001130726.3 | NP 001124198.1 |
| containing 149 | | |
| (CCDC149) | | |
| Lymphocyte antigen 96 | NM_015364.3 | NP 056179.2 |
| (LY96) | | |
| Heparanase (HPSE) | NM_001098540.2 | NP 0041092010.1 |
| DNA damage inducible | NM_019058.3 | NP 061931.1 |
| transcript 4 (DDIT4) | | |
| E2F transcription factor 3 | NM_001243076.2 | NP 001230005.1 |
| (E2F3) | | |
| ADAM metallopeptidase | NM_001261464.1 | NP 001248393.1 |
| domain 15 (ADAM15) | | |
| Succinate dehydrogenase | NM_020186.2 | NP 064571.1 |
| complex assembly factor | | |
| 3 (ACN9) | | |
| Transmembrane protein | NM_001101311.1 | NP 001094781.1 |
| 176B (TMEM176B) | | |
| Baculoviral IAP repeat | NM_001166.4 | NP 001157.1 |
| containing 2 (BIRC2) | | |
| Adhesion G protein- | NM_001271052.1 | NP 001257981.1 |
| coupled receptor E2 | | |
| (EMR2) | | |
| Dual specificity | NM_004417.3 | NP 004408.1 |
| phosphatase 1 (DUSP1) | | |
| Heat shock protein family | NM_005346.4 | NP 005337.2 |
| A 1B (HSPA1B) | | |
| Ribonuclease A family | NM_002934.2 | NP 002925.1 |
| member 2 (RNASE2) | | |
| Isopentenyl-diphosphate | NM_001317955.1 | NP 001304884.1 |
| delta isomerase 1 (IDI1) | | |
| Short coiled-coil protein | NM_001153446.1 | NP_001146918.1 |
| (SCOC) | | |
| Family with sequence | NM_001193522.1 | NP 00118045.1 |
| similarity 65, member A | | |
| (FAM65A) | | |
| CD14 molecule (CD14) | NM_000591.3 | NP_000582.1 |
| F2R like trypsin receptor | NM_005242.5 | NP_005233.3 |
| 1 (F2RL1) | | |
| Protein-L-isoaspartate | NM_001286782.1 | NM_001273711.1 |
| (D-aspartate) O- | | |
| methyltransferase domain | | |
| containing 1 (PCMTD1) | | |
| SMEK homolog 2, | NM_001122964.2 | NP 001116436.2 |
| suppressor of mek1 | | |
| (SMEK2) | | |
| Serum deprivation | NM_004657.5 | NP_004648.1 |
| response (SDPR) | | |

The early expression levels of top 25 transcripts identified by GA/kNN displayed a strong ability to differentiate between patients who later developed severe HARM and patients who did not using kNN in leave one out cross validation; a combination of just the top ten ranked transcripts (LAIRD, 1L8, CXCL5, RBP7, CCDC149, LY96, HPSE, DDIT4, E2F3, and ADAM15) were able to identify 94% of subjects correctly with a sensitivity of 88% and a specificity of 100% (FIG. 1B).

When comparing the early expression levels of the top ten transcripts between groups, all transcripts appear to be differentially expressed, however, the levels of statistical significance were modest in most cases (FIG. 1C). This suggests that in isolation, the individual transcripts would not be diagnostically robust. However, the combined predictive ability of these transcripts can be evident when their expression levels are plotted on a continuum for each individual subject; the overall pattern of expression across the top ten ranked genes can be strikingly different between patients who later developed post-stroke severe HARM and patients who did not (FIG. 1D).

The overall pattern of differential expression of the top ten transcripts between patients in the severe HARM group relative to the mild harm group remains similar when the severe HARM group can be stratified based administration on rtPA (see Table 4), suggesting that the expression levels of genes identified by GA/kNN may not be dramatically different between subjects who received rTPA and those who did not. Table 4 shows the effects of rTPA on differential expression of top ranked genes. Differential expression of the top ten genes between patients in the mild HARM group and all patients in the severe HARM group, patients in the severe HARM group who were not administered rTPA, and patients in the severe HARM group who were administered rTPA. Fold differences are reported relative to mild HARM.

TABLE 4

| | Combined (n = 8) | | No TPA (n = 4) | | TPA (n = 4) | |
|---|---|---|---|---|---|---|
| Transcript | Fold | p | Fold | p | Fold | p |
| LAIR2 | 1.61 | 0.012* | 1.37 | 0.162 | 1.86 | 0.005* |
| IL8 | 2.06 | 0.002* | 2.21 | 0.002* | 1.91 | 0.028* |
| CXCL5 | 1.38 | 0.037* | 1.50 | 0.050* | 1.27 | 0.144 |
| RBP7 | 1.49 | 0.048* | 1.34 | 0.044 | 1.64 | 0.068 |
| CCDC149 | −1.36 | 0.005* | −1.42 | 0.028* | −1.32 | 0.054 |
| LY96 | 1.79 | 0.004* | 1.76 | 0.027* | 1.82 | 0.022* |
| HPSE | −1.41 | 0.016* | −1.22 | 0.167 | −1.66 | 0.007* |
| DDIT4 | 1.72 | 0.055 | 1.38 | 0.173 | 2.06 | 0.024* |
| E2F3 | 1.51 | 0.069 | 1.19 | 0.162 | 1.82 | 0.031* |
| ADAM15 | −1.43 | 0.036* | −1.34 | 0.176 | −1.52 | 0.039* |

*SIGNIFICANT

One objective is to use GA/kNN to identify a pattern of gene expression in peripheral blood present during the acute phase of care which may be used to predict the development of post-stroke BBB disruption. In this preliminary analysis, GA/kNN was able to identify an early pattern of differential expression which proved robust in its ability to predict HARM at two days post-injury. The 10 marker panel identified herein appeared to outperform a majority of biomarkers which have been previously evaluated for their ability to predict post-stroke disruption of the BBB.

Previous studies which have attempted to identify biomarkers predicative of post-stroke BBB disruption have used the presence of hemorrhage as the criteria used to identify patients with a disrupted BBB. In this study, the presence of severe HARM was used. An approach as presented herein may be superior; the changes in BBB permeability which can be identified with HARM may be more minute than those which are required to develop hemorrhage, thus the markers described here may be more sensitive. In addition, the cerebrovascular events which can be associated with HARM preclude those which can be required to develop hemorrhage, therefore it is possible that the markers identified in this study can be detectable earlier in pathophysiology.

Several prior studies have looked to identify circulating plasma proteins which can be associated with hemorrhagic transformation; for the most part, these studies have targeted proteins which can be either involved in the breakdown of the BBB or released as a result. Such proteins include matrix metalloproteases, tight junctional proteins, and proteins which can be largely specific to the cells of the CNS. The most promising of these proteins has proven to be s100b, a calcium binding protein which can be expressed predominantly by the glial cells of the CNS. While multiple reports have agreed that circulating s100b can be elevated early in ischemic stroke patients who later undergo hemorrhagic transformation, studies targeting s100b have not demonstrated levels of diagnostic robustness which suggest it could be a clinically useful biomarker. In the largest clinical study which evaluated the ability of s100b levels to identify patients at risk for hemorrhagic transformation, s100b was only able to identify such patients with 92.9% sensitivity and 48.1% specificity. The panel of markers (biomarkers) which are identified herein outperform the majority of protein based biomarkers which have been previously evaluated for their ability to predict post-stroke BBB disruption using different criteria to classify disruption of the blood brain barrier.

One previous study has used RNA expression profiling to identify potential transcriptional biomarkers which could be used to predict hemorrhagic transformation. In this study, a panel of six gene products was identified whose expression levels showed the ability to discriminate between 11 ischemic stroke patients with hemorrhagic transformation and 33 without hemorrhagic transformation with 72.7% sensitivity and 93.9% specificity in a discovery cohort, and between 5 ischemic stroke patients with hemorrhagic transformation and 47 without hemorrhagic transformation with 80% sensitivity and 72% specificity in a separate validation cohort. The marker panel identify herein outperform this previously identified panel in terms of identifying post-stroke blood brain barrier disruption. Interestingly, none of the six markers identified in this previous study were identified in the top 10 markers in this analysis. However, one of these six previously identified markers (IRAK3) was ranked as the 35th most predicative by GA/kNN in this analysis.

### Example 6: Functional Classification Enrichment Analysis

Functional classification enrichment analysis was performed using version 6.7 of DAVID. The top 25 genes ranked by GA/kNN were submitted as a query list and all genes with expression levels detected via microarray were submitted as background. Default settings were used to identify associated Kyoto Encyclopedia of Genes and Genomes (KEGG) pathway database terms and Gene Ontology Consortium database terms which were enriched in the query list over background.

Functional classification enrichment analysis revealed that the top 25 transcripts most predictive for HARM identified by GA/kNN are up to 54 fold enriched for genes involved in chemotaxis and locomotory behavior over background (see FIG. 2), suggesting that early differential expression of genes involved in cellular migration by leukocytes may be a predictor of post-stroke BBB disruption.

Interestingly, functional annotation enrichment analysis suggested that the peripheral blood transcripts identified as most predictive of post-stroke BBB disruption were enriched for gene products which play a role in cellular migration/chemotaxis. This observation is logical from a pathophysiological perspective in that peripheral immune cells, specifically those of myeloid origin, migrate into the brain parenchyma in response to ischemic insult, which can be a process which leads to disruption of the blood brain barrier. Three of the four genes identified in this analysis which can be involved in these processes were upregulated in patients who later developed severe HARM, while one gene was downregulated.

The expression levels of the genes encoding for the chemokines interleukin-8 (IL-8) and chemokine ligand 5 (CXCL5) were both upregulated in patients who later developed severe HARM. Both chemokines can be produced by monocytes and macrophages, and induce a strong chemotactic response in neutrophils and other granulocytes. Increased protein levels of both IL-8 and CXCL5 have been reported in the CSF of stroke patients, suggesting they may play a major role in the recruitment of peripheral immune cells into the CNS following ischemic injury. In support of these observations, a recent study demonstrated that genetic ablation of CXCL5 in myeloid derived blood cells dramatically reduces neutrophil infiltration and BBB disruption in an animal model of ischemic stroke. The remaining upregulated gene, F2RL1, encodes a g-protein coupled receptor known as proteinase activated receptor 2 (PAR2) which can be found on peripheral blood granulocytes33. PAR2 can be activated specifically by trypsin and factor X, and plays a significant role in initiating endothelial rolling and tissue infiltration in neutrophils. Based in their collective role in promoting neutrophil migration and invasion, it is rational that the early upregulation of these transcripts could drive neutrophil-mediated BBB disruption in the context of stroke. The one gene downregulated in patients who later developed severe HARM associated with chemotaxis and migration, RNASE2, encodes for a ribonuclease-A superfamily protein known as eosinophil-derived neurotoxin (EDN). EDN can be produced by neutrophils and other granulocytes and induces chemotaxis specifically in dendritic cells. Suppression of the peripheral adaptive immune system can occur in response to stroke, most likely as a defense mechanism to prevent an autoimmune response driven by the activation of adaptive immune cells by CNS antigens upon disruption of the BBB. Thus, it is logical that it would be beneficial to downregulate the expression of molecules which would aid in the recruitment of professional antigen presenting cells to the brain parenchyma during the development of BBB disruption.

### Example 7: Statistical Analysis

Statistical analysis was performed using the SPSS statistical software package (IBM, Chicago, Ill.). Chi squared analysis was used for comparison of dichotomous variables while student t-test was used for the comparison of continuous variables. The level of significance was established at 0.05 for all statistical testing.

### Discussion

Collectively, the results of this analysis indicate a highly accurate RNA-based biomarker panel which can identify patients at risk for post-stroke BBB disruption. If such an assay were to be implemented, it could provide invaluable diagnostic information which could be used to improve clinical decision making in the acute phase of care. These results suggest that early expression of molecules which promote peripheral innate immune cell migration may constitute a risk factor for development of post-stroke BBB disruption. Further exploration into this phenomenon could lead to therapeutic interventions aimed to reduce post-injury progression of BBB damage and improve outcome.

### Exemplary Study 2

Experimental Design:

Peripheral blood samples were obtained from acute ischemic patients within 24 hours of symptom onset, before the administration of TPA, and BBB permeability was assessed by level of hyperintense acute reperfusion marker (HARM) on MRI two days post-injury. Peripheral blood RNA expression profiles were generated for 8 patients exhibiting severe harm and 8 patients exhibiting mild harm using microarray, and GA/kNN was applied to rank transcripts based on their ability to discriminate between harm categories. Bioinformatic analysis of functional classification enrichment was then used to identify the biological significance of the identified transcripts.

### Example 8—Patient Selection

Demographic and Clinical Characteristics:

Patients in the mild HARM and severe HARM groups were well matched in terms of cardiovascular disease risk factors, comorbidities, and medication status. Stroke severity via NIHSS was greater at ED admission in patients in the severe HARM group than in the mild HARM group, however, almost all patients presented with relatively low NIHSS scores. Subjects were well matched on most variables, however NIHSS and rtPA are significantly different between groups (see Table 2 above).

### Example 9: Blood Collection and RNA Extraction

Peripheral whole blood samples were collected via PAXgene RNA tubes (Qiagen, Valencia, Calif.) and stored at −80° C. until RNA extraction. Total RNA was extracted via the PreAnalytiX PAXgene blood RNA kit (Qiagen). Quantity and purity of isolated RNA was determined via spectrophotometry (NanoDrop, Thermo Scientific, Waltham, Mass.) and quality of RNA was confirmed by gel electrophoresis.

### Example 10: RNA Amplification and Microarray

RNA was amplified and biotinylated using the TotalPrep RNA amplification kit (Applied Biosystems, Grand Island, N.Y.). The TotalPrep RNA amplification kit was employed to generate biotinylated, amplified RNA for hybridization to the arrays. The procedure consisted of reverse transcription with an oligo (dT) primer and a reverse transcriptase designed to produce higher yields of first strand cDNA. The cDNA underwent a second strand synthesis and clean up to become a template for in vitro transcription. The in vitro transcription resulted in biotin labeled antisense cRNA copies of each mRNA in a sample.

Samples were hybridized to HumanRef-8 expression bead chips (Illumina, San Diego, Calif.) containing probes for transcripts originating from over 10,000 genes and scanned using the Illumina BeadStation. The expression beadchips can be constructed by introducing oligonucleotide bearing 3-micron beads into microwells etched into the surface of a slide-sized silicon substrate. The beads self-assemble onto the beadchips resulting in an average of 30-fold redundancy of every full-length oligonucleotide. After random bead assembly, 29-mer address sequences present on each bead can be used to map the array, identifying the location of each bead.

Raw probe intensities were background subtracted, quantile normalized, and then summarized at the gene level using Illumina GenomeStudio. Sample labeling, hybridization, and scanning were performed per standard Illumina protocols.

### Example 11: GA/kNN Analysis

Normalized microarray data were filtered based on absolute fold difference between HARM categories; Genes exhibiting a greater than 1.5 absolute fold difference in expression between HARM categories were used for analysis. Filtered gene expression was z-transformed and GA/kNN analysis was performed using publically available source code developed by Leping Li et al, 2001. One-thousand near-optimal solutions were collected per sample using five nearest neighbors, majority rule, a chromosome length of 5, and a termination cutoff of 0.875. Leave one out cross validation was performed using the top 25 ranked variables.

The top 25 most predictive transcripts ranked by GA/kNN, as indicated by the number of times selected in a near optimal solution, are listed in FIG. 1A.

The fold differences between groups and statistical differences are depicted in FIG. 1C.

The top 10 transcripts used in combination were able to correctly classify 15 of the 16 samples (93.8%) using kNN in leave one out cross validation (FIG. 1B). On average, any combination of the top 5 ranked or more transcripts was able to correctly classify 14/16 samples.

When the expression levels of the top 10 ranked variables are plotted for each individual subject, it is clear that the overall pattern of expression can be distinctly different between groups (FIG. 1D). It is clear that the overall pattern of expression can be more diagnostically powerful than the expression levels of any given transcript on its own.

The overall pattern of expression of the top 10 transcripts in the severe harm group relative to the mild harm group remains similar when the severe HARM group is stratified based administration on rtPA.

### Example 12: Functional Classification Enrichment Analysis

The DAVID bioinformatics resource was used to identify functional categories of genes statistically enriched along the top 25 most predictive variables identified by GA/kNN. DAVID was used to query the NCBI gene ontology database, Panther molecular process database, and Kegg pathway database using default parameters as described by D W Haung et al, 2009.

Functional classification enrichment analysis reveals that the top 25 most predictive transcripts are enriched for genes involved in chemotaxis and locomotory behavior (FIG. 2), suggesting that early expression of chemoattractant molecules by leukocytes may be a predictor of poststroke BBB disruption.

### Example 13: Comparison of Mild HARM and Hemorrhagic Transformation

The expression levels of the ten genes identified in this study was compared to the mild HARM group and 5 additional patients who underwent hemorrhagic transformation (Table 5). In this analysis, we observed an identical pattern of differential expression as we did when comparing the mild HARM group to the severe HARM group (Table 6), suggesting that these markers may likely not be HARM specific, and should be able to predict more dramatic cases of blood brain barrier disruption such as hemorrhagic transformation as well. Table 5 sets forth demographic and clinical characteristics of hemorrhagic transformation patients.

TABLE 5

| | MILD | HEMORRHAGE | | |
|---|---|---|---|---|
| | HARM | (n = 8) | STAT (df) | P |
| Age (mean ± SD) | 72.9 ± 11.8 | 66.2 ± 15.6 | t = 0.88 (11) | 0.396 |
| NIHSS (mean ± SD) | 1.0 ± 2.1 | 6.6 ± 7.6 | t = −2.01 (11) | 0.069 |
| Male n (%) | 3 (37.5) | 2 (40.0) | χ² = 0.01 (1) | 0.928 |
| Female n (%) | 5 (62.5) | 3 (60.0) | χ² = 0.01 (1) | 0.928 |
| Family history of stroke n (%) | 4 (50.0) | 2 (40.0) | χ² = 0.12 (1) | 0.725 |
| Hypertension n (%) | 5 (62.5) | 3 (60.0) | χ² = 0.01 (1) | 0.928 |
| Diabetes n (%) | 1 (12.5) | 1 (20.0) | χ² = 0.13 (1) | 0.715 |
| Atrial fibrillation n (%) | 1 (12.5) | 0 (0.00) | χ² = 0.67 (1) | 0.412 |
| Myocardial infarction n (%) | 2 (25.0) | 1 (20.0) | χ² = 0.04 (1) | 0.835 |
| Dyslipidemia n (%) | 5 (62.5) | 4 (80.0) | χ² = 0.29 (1) | 0.590 |
| Previous stroke n (%) | 0 (0.00) | 0 (0.00) | χ² = 0.00 (1) | 1.000 |
| Hypertension medication n (%) | 5 (62.5) | 4 (80.0) | χ² = 0.44 (1) | 0.506 |
| Diabetes medication n (%) | 1 (12.5) | 0 (0.00) | χ² = 0.67 (1) | 0.412 |
| Cholesterol medication n (%) | 5 (62.5) | 2 (40.0) | χ² = 0.63 (1) | 0.428 |
| Anticoagulant or antiplatelet n (%) | 5 (62.5) | 2 (40.0) | χ² = 0.63 (1) | 0.428 |
| rtPA n (%) | 0 (0.00) | 3 (60.0) | χ² = 3.70 (1) | 0.046* |
| Current smoker n (%) | 0 (0.00) | 0 (0.00) | χ² = 0.00 (1) | 1.000 |
| Previous smoker n (%) | 2 (25.0) | 2 (40.0) | χ² = 0.33 (1) | 0.588 |

*SIGNIFICANT

Table 6 sets forth the pattern of expression in hemorrhagic transformation.

TABLE 6

| Transcript | Fold | P |
|---|---|---|
| LAIR2 | 1.4 | 0.12 |
| IL8 | 1.4 | 0.14 |
| CXCL5 | 1.2 | 0.21 |
| RBP7 | 1.5 | 0.07 |
| CCDC149 | −1.2 | 0.27 |
| LY96 | 1.5 | 0.11 |
| HPSE | −1.3 | 0.06 |
| DDIT4 | 1.7 | 0.07 |
| E2F3 | 1.6 | 0.05* |
| ADAM15 | −1.4 | 0.02* |

*SIGNIFICANT

Differential expression of the top ten genes identified by GA/kNN as predictive of post-stroke BBB disruption between the mild HARM group and hemorrhagic transformation patients, reported as fold difference relative to mild HARM.

It will be appreciated that this disclosure provides a method for determining blood brain barrier disruption or hemorrhagic transformation (brain bleeding) or risk of blood brain barrier disruption and hemorrhagic transformation in a patient presenting with symptoms characteristic of a stroke or at risk of having a stroke or other neurological disease, that can comprise obtaining a biological sample from the patient, and contacting the biological sample with a detection means to detect the presence of the identified biomarker profile.

The methods described herein can produce a marker or predictor of blood brain barrier disruption and hemorrhagic transformation in human having ischemic stroke; a marker or predictor of blood brain barrier disruption and hemorrhagic transformation in other neurological diseases such as for example, but not limited to, multiple sclerosis, Alzheimer's disease, migraine, epilepsy, and traumatic brain injury; as a therapeutic target for stroke, brain injury treatment, and neurological disease treatment; a therapeutic target for therapeutic disruption of the blood brain barrier for brain cancers; a marker of brain tissue injury; a prognostic indicator of health outcome following neurologic injury; and a marker to be used for stratification of risk for treatment decision making in stroke or brain injuries.

While some embodiments described herein have been shown and described herein, such embodiments are provided by way of example only. Numerous variations, changes, and substitutions will now occur to those skilled in the art without departing from the disclosure provided herein. It should be understood that various alternatives to the embodiments described herein can be employed in practicing the methods described herein.

What is claimed is:

1. A method comprising:
(a) performing, using a computer processor, an algorithm on a biological sample from a subject to generate a fitness score for a chromosome of data, wherein the subject was previously diagnosed with a blood-brain barrier disruption as determined by contrast MRI, wherein the computer processor executes instructions to perform the functional classification enrichment analysis;
(b) performing multiple iterations of the algorithm until the fitness score exceeds a termination cutoff; and
(c) compiling a profile, wherein the profile comprises at least one biomarker that is involved in chemotaxis as determined by functional classification enrichment analysis.

2. The method of claim 1, wherein the algorithm comprises a machine learning algorithm.

3. The method of claim 2, wherein the machine learning comprises a deep learning algorithm.

4. The method of any one of claims 1-3, wherein the algorithm comprises analyzing an initial panel of at least about 10,000 genes.

5. The method of claim 2, wherein the machine learning algorithm comprises genetic algorithm k-neared neighbors.

6. The method of any one of claims 1-5, wherein the termination cutoff is about 0.85.

7. The method of any one of claims 1-6, wherein the chromosome of data has a chromosome length of at least about 10.

8. A system for detecting a blood-brain barrier disruption in a subject, the system comprising:
(a) a memory that stores executable instructions; and
(b) a computer processor that executes instructions to perform the method of any one of claims 1-7.

9. The system of claim 8, further comprising an integrated storage device.

10. The system of claim 8, wherein the system is configured to communicate with a database for performing functional classification enrichment analysis.

11. A kit for assessing blood-brain barrier disruption in a subject, the kit comprising:
(a) a probe for measuring a presence of a panel of biomarkers in a biological sample obtained from the subject, wherein the panel of biomarkers comprises a nucleic acid, and wherein the probe can hybridize to the nucleic acid in the biological sample; and
(b) a detecting reagent to examine hybridization of the probe to the nucleic acid, wherein the panel of biomarkers comprises one or more biomarkers selected from the group consisting of: RBP7, CCDC149, DDIT4, E2F3, and ADAM15.

12. The kit of claim 11, further comprising instructions for use.

13. The kit of claim 11, wherein the panel of biomarkers comprises at least two biomarkers.

14. The kit of claim 13, wherein the panel of biomarkers comprises RBP7, CCDC149, DDIT4, E2F3, and ADAM15.

15. The kit of any one of claims 13-15, wherein the panel of biomarkers further comprises LAIR2, IL-8, CXCL5, LY96, HPSE, ACN9, TMEM176B, BIRC2, EMR2, DUSP1, HSPA1B, RNASE2, IDI1, SCOC, FAM65A, CD14, F2RL1, PCMTD1, SMEK2, or SDPR.

16. The kit of claim 15, wherein the panel of biomarkers comprises LAIR2, IL-8, CXCL5, LY96, and HPSE.

17. The kit of claim 15, wherein the panel of biomarkers comprises LAIR2, RBP7, CCDC149, DDIT4, E2F3, ADAM15, LAIR2, IL-8, CXCL5, LY96, and HPSE.

18. The kit of any one of claims 11-17, further comprising a communication medium that is configured to communicate hybridization of the probe to the nucleic acid.

19. The kit of claim 18, wherein the communication medium is an electronic medium.

20. A method comprising:
(a) determining a presence of a panel of biomarkers in a biological sample obtained from a subject using an assay, wherein the subject is a subject having blood brain barrier disruption or suspected of having blood brain barrier disruption, wherein the panel of biomarkers comprises one or more biomarkers selected from the group consisting of: RBP7, CCDC149, DDIT4, E2F3, and ADAM15; and
(b) comparing the presence of the panel of biomarkers in the biological sample obtained from the subject to a reference derived from one or more control samples.

21. The method of claim 20, wherein the panel of biomarkers comprises at least two biomarkers.

22. The method of claim 20 or 21, wherein the panel of biomarkers comprises RBP7, CCDC149, DDIT4, E2F3, and ADAM15.

23. The method of any one of claims 20-22, wherein the panel of biomarkers further comprises LAIR2, IL-8, CXCL5, LY96, HPSE, ACN9, TMEM176B, BIRC2, EMR2, DUSP1, HSPA1B, RNASE2, IDI1, SCOC, FAM65A, CD14, F2RL1, PCMTD1, SMEK2, or SDPR.

24. The method of claim 23, wherein the panel of biomarkers comprises LAIR2, RBP7, CCDC149, DDIT4, E2F3, ADAM15, LAIR2, IL-8, CXCL5, LY96, and HPSE.

25. The method of claim 20, wherein the one or more biomarkers comprise ribonucleic acid.

26. The method of claim 20, wherein the one or more biomarkers comprise a gene that is involved in chemotaxis.

27. The method of any one of claims 20-26, wherein the subject is suspected of having a stroke.

28. The method of any one of claims 20-27, wherein the one or more control samples are from one or more control subjects.

29. The method of claim 28, wherein the one or more control subjects are stroke subjects.

30. The method of claim 28, wherein the stroke subjects are ischemic stroke subjects.

31. The method of claim 28, wherein the one or more control subjects are nonstroke subjects.

32. The method of any one of claims 28-31, wherein the reference was determined after the one or more control subjects were administered a contrast agent.

33. The method of claim 32, wherein the contrast agent comprises a gadolinium-based contrast agent.

34. The method of claim 33, wherein the gadolinium-based contrast agent comprises gadolinium-diethylene triamine penta-acetic acid (Gd-DTPA).

35. The method of any one of claims 28-34, wherein the one or more control subjects were diagnosed with a blood brain barrier disruption or a risk of a blood-brain barrier disruption.

36. The method of any one of claims 20-35, wherein the presence comprises a level of the panel of biomarkers.

37. The method of any one of claims 20-36, further comprising assessing a blood brain barrier disruption in the subject.

38. The method of claim 37, wherein the assessing comprises determining a presence of a blood brain barrier disruption.

39. The method of claim 37, wherein the assessing comprises determining a risk of a blood brain barrier disruption.

40. The method of claim 37, wherein the assessing comprises determining an absence of a blood brain barrier disruption.

41. The method of any one of claims 38-40, wherein the panel of biomarkers is at least about 1.5 fold higher in the subject relative to the reference.

42. The method of any one of claims 38-40, wherein the panel of biomarkers is at least about 1.5 fold lower in the subject relative to the reference.

43. The method of any one of claims 37-42, wherein the assessing is performed with a sensitivity of at least about 90%.

44. The method of any one of claims 37-42, wherein the assessing is performed with a specificity of at least about 96%.

45. The method of any one of claims 20-44, wherein the assay comprises hybridizing a probe to the panel of biomarkers or a portion thereof.

46. The method of claim 45, further comprising detecting the hybridizing.

47. The method of claim 45 or 46, wherein the probe is a fluorescent probe.

48. The method of any one of claims 45-47, further comprising communicating a result through a communication medium when the probe hybridizes with the panel of biomarkers or a portion thereof.

49. The method of claim 48, wherein the communication medium comprises an electronic medium.

50. A method comprising: determining a presence of a panel of biomarkers in a biological sample obtained from a subject having stroke or suspected of having stroke using an assay, wherein the presence of the panel biomarkers is indicative of hyperintense acute reperfusion marker (HARM) on fluid-attenuated inversion recovery (FLAIR) MRI when a contrast agent is administered to said subject having stroke or suspected of having stroke; and wherein the panel of biomarkers comprises one or more biomarkers selected from the group consisting of: LAIR2, RBP7, CCDC149, DDIT4, E2F3, and ADAM15.

51. The method of claim 50, wherein the panel of biomarkers comprises at least two biomarkers.

52. The method of claim 50 or 51, wherein the panel of biomarkers comprises LAIR2, RBP7, CCDC149, DDIT4, E2F3, and ADAM15.

53. The method of any one of claims 50-52, wherein the panel of biomarkers further comprises IL-8, CXCL5, LY96, HPSE, ACN9, TMEM176B, BIRC2, EMR2, DUSP1, HSPA1B, RNASE2, IDI1, SCOC, FAM65A, CD14, F2RL1, PCMTD1, SMEK2, or SDPR.

54. The method of claim 53, wherein the panel of biomarkers comprises IL-8, CXCL5, LY96, and HPSE.

55. The method of claim 53, wherein the panel of biomarkers comprises LAIR2, RBP7, CCDC149, DDIT4, E2F3, ADAM15, IL-8, CXCL5, LY96, and HPSE.

56. The method of any one of claims 50-55, wherein the stroke is an ischemic stroke.

57. The method of any one of claims 50-57, wherein the contrast agent comprises a gadolinium-based contrast agent.

58. The method of claim 57, wherein the gadolinium-based contrast agent comprises gadolinium-diethylene triamine penta-acetic acid (Gd-DTPA).

59. The method of any one of claims 50-58, wherein the HARM is severe HARM.

60. The method of claim 59, wherein severe HARM is indicative of a blood-brain barrier disruption.

61. The method of any one of claims 50-60, wherein the presence comprises a level of the panel of biomarkers.

62. The method of any one of claims 50-61, further comprising comparing the presence of the panel of biomarkers to a reference.

63. The method of claim 62, wherein the reference is derived from one or more control samples.

64. The method of claim 62 or 63, wherein the panel of biomarkers is at least about 1.5 fold higher in the subject relative to the reference.

65. The method of claim 62 or 63, wherein the panel of biomarkers is at least about 1.5 fold lower in the subject relative to the reference.

66. The method of any one of claims 50-65, further comprising administering a therapeutic to the subject.

67. The method of any one of claims 50-66, wherein the assay comprises hybridizing a probe to the panel of biomarkers or portions thereof.

68. The method of claim 67, further comprising detecting the hybridizing.

69. The method of claim 67 or 68, wherein the probe is a fluorescent probe.

70. The method of any one of claims 67-69, further comprising communicating a result through a communication medium when the probe hybridizes with the panel of biomarkers or a portion thereof.

71. The method of claim 70, wherein the communication medium comprises an electronic medium.

72. A method comprising:
(a) determining a presence of a panel of biomarkers in a biological sample obtained from a subject using an assay; thereby determining a profile for the subject; and
(b) assessing a blood brain barrier disruption in the subject, wherein the assessing is performed with a sensitivity of at least about 90% and a specificity of at least about 96%.

73. The method of claim 72, wherein the panel of biomarkers comprises at least two biomarkers.

74. The method of claim 72 or 73, wherein the panel of biomarkers comprises one or more biomarkers selected from the group consisting of: LAIR2, IL-8, CXCL5, RBP7, CCDC149, LY96, DDIT4, HPSE, E2F3, ADAM15, ACN9, TMEM176B, BIRC2, EMR2, DUSP1, HSPA1B, RNASE2, IDI1, SCOC, FAM65A, CD14, F2RL1, PCMTD1, SMEK2, and SDPR.

75. The method of any one of claims 72-74, wherein the panel of biomarkers comprises LAIR2, RBP7, CCDC149, DDIT4, E2F3, ADAM15, IL-8, CXCL5, LY96, and HPSE.

76. The method of any one of claims 72-75, wherein the panel of biomarkers comprises ribonucleic acid.

77. The method of any one of claim 72-75, wherein the biomarkers comprise a gene that is involved in chemotaxis.

78. The method of any one of claims 72-77, further comprising comparing the profile to a reference.

79. The method of claim 78, wherein the one or more control samples are from one or more control subjects.

80. The method of claim 79, wherein the reference was determined after the one or more control subjects were administered a contrast agent.

81. The method of claim 80, wherein the contrast agent comprises a gadolinium-based contrast agent.

82. The method of claim 81, wherein the gadolinium-based contrast agent comprises gadolinium-diethylene triamine penta-acetic acid (Gd-DTPA).

83. The method of any one of claims 72-82, wherein the assessing comprises determining a presence of the blood brain barrier disruption.

84. The method of any one of claims 72-82, wherein the assessing comprises determining a risk of the blood brain barrier disruption.

85. The method of any one of claims 72-82, wherein the assessing comprises determining an absence of the blood brain barrier disruption.

86. The method of any one of claims 20-85, wherein the biological sample comprises whole blood, peripheral blood, or cerebrospinal fluid.

87. The method of any one of claims 20-85, wherein the biological sample comprises cell-free nucleic acids.
